(19) United States
(12) Patent Application Publication
(10) Pub. No.: US 2007/0069197 A1
(43) Pub. Date: Mar. 29, 2007

(54) Monomers, oligomers and polymers of 2-functionalized and 2,7-difunctionalized carbzoles

(75) Inventor: Mario Leclerc, (US)

Correspondence Address: FULBRIGHT & JAWORSKI L.L.P., 600 CONGRESS AVE., SUITE 2400, AUSTIN, TX, 78701, US

(73) Assignee: Universite Laval, Quebec (CA)

(21) Appl. No.: 10/568,303

(22) PCT Filed: Aug. 16, 2004
(86) PCT No.: PCT/CA04/01509

§ 371 Date: Oct. 20, 2006

Related U.S. Application Data
(60) Provisional application No. 60/495,113, filed on Aug. 15, 2003.

(51) Int. Cl. H01L 51/00; C07D 209/82

(52) U.S. Cl. 257/40; 548/444; 528/423; 257/E51.02; 548/414

(57) ABSTRACT

The present invention relates to 2-functionalized and 2,7-difunctionalized carbazoles and 2,7-carbazolenevinylene oligomers and polymers. More specifically, the present invention relates to a compound of Formula (I): wherein R¹ is selected from the group consisting of H, alkyl, and aryl; and wherein R² and R³ are independently selected from the group consisting of H, alkyl, formyl, hydroxymethyl, trityloxymethyl, acetonitrile, chloromethyl, methylphosphonate, methyltriphenylphosphonium and vinyl. The oligomers and polymers are used in field-effect transistors, light-emitting devices such as light-emitting diodes, and solar cells.

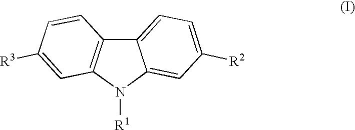

### FIELD OF THE INVENTION

The present invention relates to a new class of organic material. More specifically, the present invention is relates to monomers, oligomers and polymers of 2 functionalized and 2,7-difunctionalized carbazoles.

### BACKGROUND OF THE INVENTION

Conjugated polymeric and oligomeric organic materials are subject to important investigations from both academic and industrial laboratories, due to their great potential for applications in light-emitting diodes, field-effect transistors, sensors, solar cells, etc.¹⁻⁷

The relatively low cost synthesis, ease of processability and the great tunability of their optical and electrical properties through chemical modification are just some of the advantages provided by organic semi-conducting materials over their inorganic counter parts.

Important developments in modern synthetic chemistry, especially the chemistry of carbon-carbon bond formation (Kumada, Stille, Yamamoto, Suzuki, Heck, and Sonogashira couplings, etc.) have allowed the synthesis of well-defined conjugated oligomers and polymers having a high degree of purity and improved physical properties in comparison to those obtained by traditional oxidative couplings. Moreover, a good understanding of the structure-property relationship, combined with the many new, highly selective synthetic methods now available, have allowed for the development of a nearly unlimited number of structures having specific properties and performances approaching those of their inorganic counterparts. Small molecules having planar structures generally lead to highly ordered solid π-π* interactions. Therefore, 2,7-carbazolenevinylene-based materials can thus be used in electronic devices requiring good charge transport properties, such as in field-effect transistors. Depending on the sought-after applications, different building blocks, such as thiophene, pyrrole, phenylene, fluorene and carbazole can be used, irrespective of their specific properties. In this regard, 2,7-carbazole-based well-defined polymers have been recently prepared.^{8,9} Their good fluorescence properties have led to the preparation and testing in light-emitting diodes of electroluminescent polymers spanning the entire visible range.^{10,11,12} The introduction of a vinylene unit into the polymer backbone is known to decrease the band gap due to the relatively low dihedral angle between the vinylene unit and a common aryl group. Consequently, medium to low band gap materials can be obtained, allowing for the preparation of a wide variety of luminescent polymers providing for green to red-light emissions.

However, the development of new building blocks for the preparation of 2,7-carbazolenevinylene-based materials remains a challenge to any chemist or physicist desirous of optimizing material performance in electronic devices requiring good charge transport properties.

The present invention seeks to meet these needs and other needs.

The present description refers to a number of documents, the content of which is herein incorporated by reference in their entirety.

### SUMMARY OF THE INVENTION

The present invention relates to 2 functionalized and 2,7-difunctionalized carbazoles as well as to methods for preparing these carbazoles. More specifically, the present invention relates to a compound of Formula I:

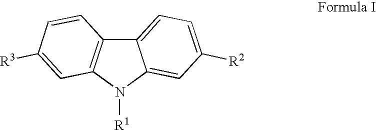

wherein R¹ is selected from the group consisting of H, alkyl, and aryl; and wherein R² and R³ are independently selected from the group consisting of H, alkyl, formyl, hydroxymethyl, trityloxymethyl, acetonitrile, chloromethyl, methylphosphonate, methyltriphenylphosphonium and vinyl.

Yet more specifically, the present invention relates to 2 functionalized and 2,7-difunctionalized carbazoles selected from the group consisting of:

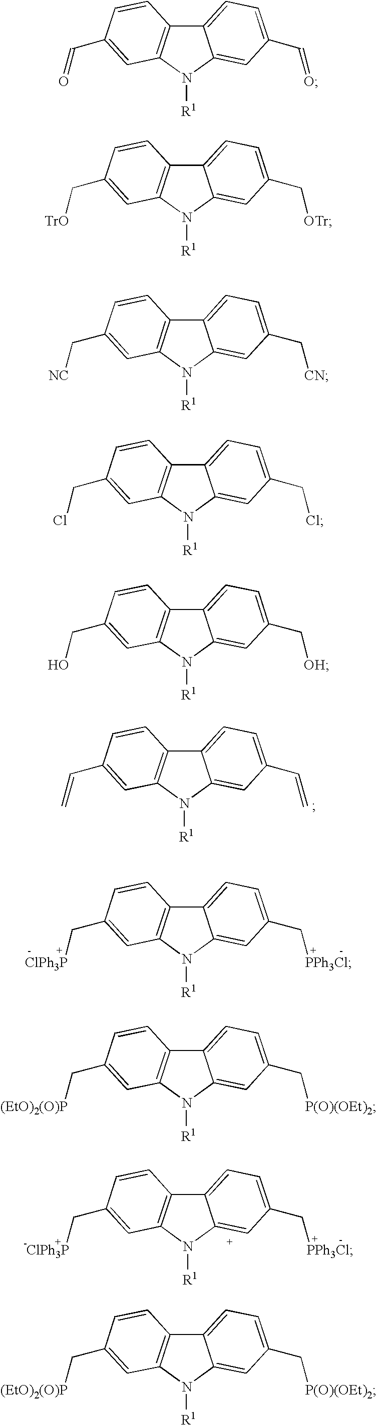

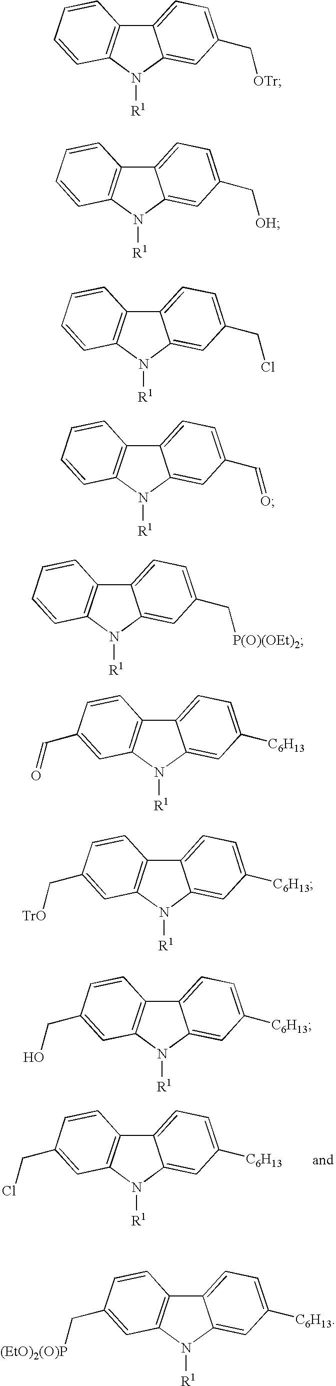

The present invention also relates to 2,7-carbazolenevinylene-based oligomers as well as to methods for preparing these oligomers.

Yet more specifically, the present invention relates to a 2,7-carbazolenevinylene-based oligomer comprising the reaction product of a first compound of Formula I and at least a second compound, the second compound being either a compound of Formula I; benzaldehyde; 5,5′-diformyl-2-2′bithiophene, 4-bromo-1,1′biphenyl; benzyl cyanide; or 1,4-bis(methylphosphonate)benzene.

In a first particular embodiment, the present invention relates to a 2,7-carbazolenevinylene-based oligomer having the formula:

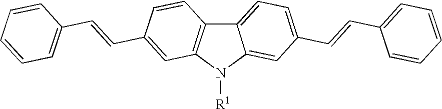

wherein R¹ is selected from the group consisting of H, alkyl, and aryl.

In a second particular embodiment, the present invention relates to a 2,7-carbazolenevinylene-based oligomer having the formula:

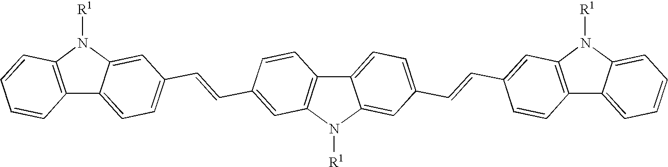

wherein R¹ is selected from the group consisting of H, alkyl, and aryl.

In a third particular embodiment, the present invention relates to a 2,7-carbazolenevinylene-based oligomer having the formula:

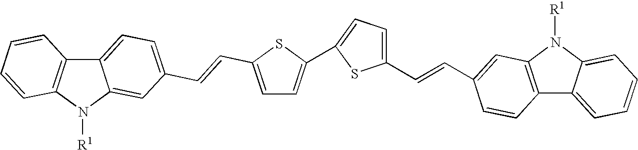

wherein R¹ is selected from the group consisting of H, alkyl, and aryl.

In a fourth particular embodiment, the present invention relates to a 2,7-carbazolenevinylene-based oligomer having the formula:

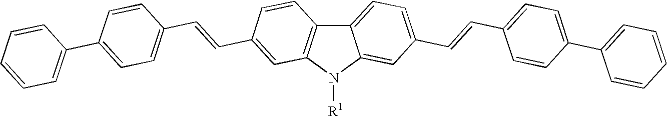

wherein R¹ is selected from the group consisting of H, alkyl, and aryl.

In a fifth particular embodiment, the present invention relates to a 2,7-carbazolenevinylene-based oligomer having the formula:

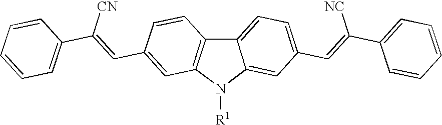

wherein R¹ is selected from the group consisting of H, alkyl, and aryl.

In a sixth particular embodiment, the present invention relates to a 2,7-carbazolenevinylene-based oligomer having the formula:

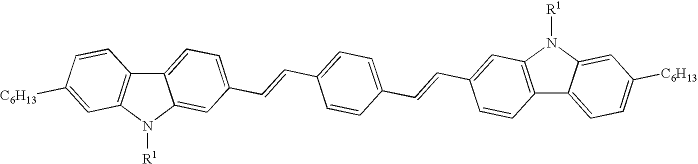

wherein R¹ is selected from the group consisting of H, alkyl, and aryl.

The present invention additionally relates to 2,7-carbazolenevinylene-based polymers as well as to methods of preparing these polymers.

Yet more specifically, the present invention relates to 2,7-carbazolenevinylene-based polymers comprising the reaction product of a compound of Formula I and optionally at least one compound selected from the group consisting of 2,5-dioctyloxy-1,4-diformylbenzene; 2,5-bis(diphenylamino)terephthaldicarboxaldehyde; {4-(2-ethylhexyloxy)-phenyl]-bis-(4′formylphenyl); 6,6′-dibromo-2,2′-bis(2″-ethylhexyloxy)-1,1′-binaphthyl; and 3-hexyl-2,5-bis(methylphosphonate)thiophene.

In a first particular embodiment, the present invention relates to a 2,7-carbazolenevinylene-based polymer having the formula:

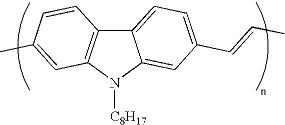

wherein “n” is an integer ranging from 5 to 100.

In a second particular embodiment, the present invention relates to a 2,7-carbazolenevinylene-based polymer having the formula:

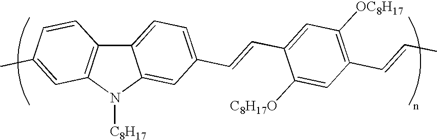

wherein “n” is an integer ranging from 5 to 100.

In a third particular embodiment, the present invention relates to a 2,7-carbazolenevinylene-based polymer having the formula:

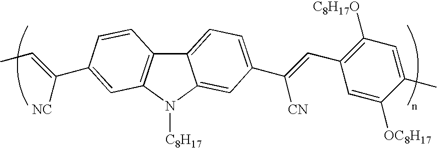

wherein “n” is an integer ranging from 5 to 100.

In a fourth particular embodiment, the present invention relates to a 2,7-carbazolenevinylene-based polymer having the formula:

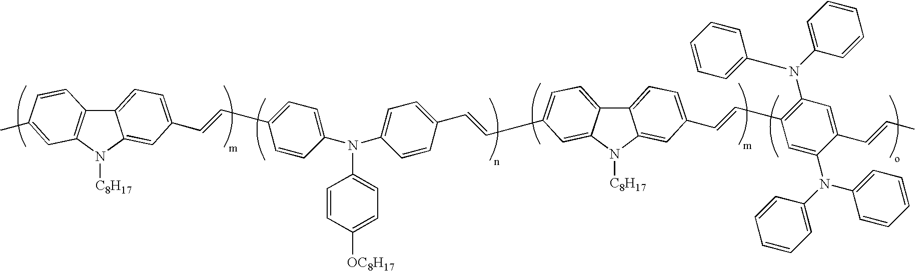

wherein “n”, “m” and “o” are integers ranging from 5 to 100.

In a fifth particular embodiment, the present invention relates to a 2,7-carbazolenevinylene-based polymer having the formula:

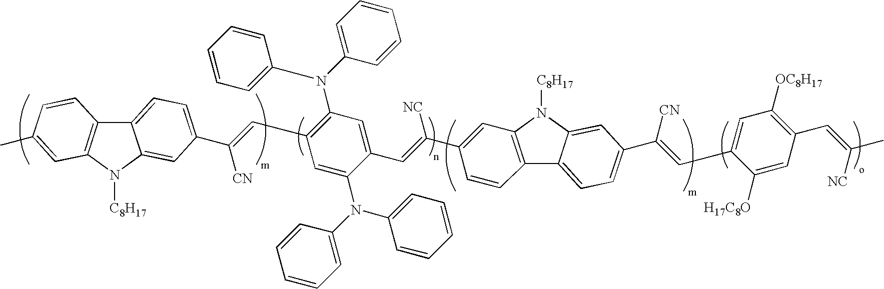

wherein “n”, “m” and “o” are integers ranging from 5 to 100.

In a sixth particular embodiment, the present invention relates to a 2,7-carbazolenevinylene-based polymer having the formula:

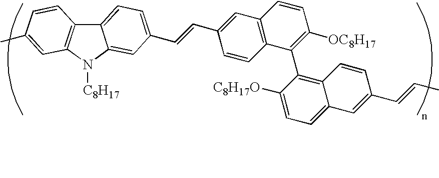

wherein “n” is an integer ranging from 5 to 100.

In a seventh particular embodiment, the present invention relates to a 2,7-carbazolenevinylene-based polymer having the formula:

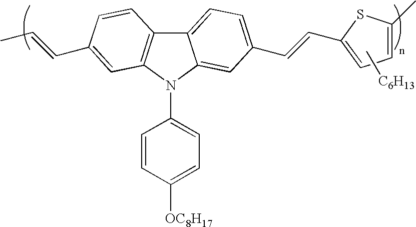

wherein “n” is an integer ranging from 5 to 100.

The present invention also relates to 2,7-carbazolenevinylene-based oligomers and polymers for use in applications including but not limited to field-effect transistors, light-emitting devices such as light-emitting diodes, and solar cells.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

In the appended drawings:

FIG. 1 illustrates the synthesis of novel 2,7-difunctionalized carbazoles;

FIG. 2 illustrates the synthesis of 2-functionalized carbazoles;

FIG. 3 illustrates the chemical structure of various oligomers;

FIG. 4 illustrates the chemical structure of various polymers;

FIG. 5 provides a schematic illustration of the polymerization yield obtained for various polymers as well as their molecular weight;

FIG. 6 provides a schematic illustration of the optical properties of various polymers;

FIG. 7 provides a schematic illustration of the optical and electrochemical properties of various oligomers; and

FIG. 8 illustrates the absorption and emission spectra of PCCVP in chloroform as well as in the solid state.

### DESCRIPTION OF ILLUSTRATIVE EMBODIMENTS

As used herein, the term “alkyl” is intended to include linear, branched and cyclic structures, as well as combinations thereof, having up to 10 carbon atoms. Non-limiting examples of alkyl groups include methyl, ethyl, propyl, isopropyl, cyclopropyl, butyl, sec-butyl, tert-butyl, cyclobutyl, pentyl, cyclopentyl; hexyl, cyclohexyl, heptyl, cycloheptyl, octyl, cyclooctyl, 2-ethylhexyl, nonyl and decyl.

As used herein, the term “alkoxy” is intended to include such alkyl groups as defined above attached to an oxygen atom. Non-limiting examples of alkyl groups include methoxy, ethoxy, propoxy, isopropoxy, cyclopropoxy, butoxy, sec-butoxy, tert-butoxy, cyclobutoxy, pentoxy, cyclopentoxy, hexyloxy, cyclohexyloxy, heptyloxy, cycloheptyloxy, octyloxy, cyclooctyloxy, nonyloxy and decyloxy.

As used herein, the term “aryl” is intended to mean an aromatic ring structure having, for example, 6-10 carbon atoms, preferably a phenyl group or a phenyl group substituted with an alkyl or alkoxy group, wherein the terms alkyl and alkoxy are as defined above.

As used herein, the term “oligomer” is intended to mean a molecule composed of a at least 2 linked monomer units; more preferably, 2 to 4 linked monomer units.

As used herein, the term “polymer” is intended to mean a molecule composed of a at least 5 linked monomer units; preferably, 5 to 500 linked monomer units, and more preferably 5 to 100 linked monomer units. It is to be understood that the polymers as described herein may be composed of different monomeric units.

### EXPERIMENTAL

Characterization: Number-average (Mₙ) and weight-average (M_{w}) molecular weights were determined by size exclusion chromatography (SEC) using an HPLC pump and a Waters UV-vis detector. A calibration curve was prepared using a series of monodispersed polystyrene standards in THF (HPLC grade, Aldrich). UV-vis absorption spectra were recorded on a Hewlett-Packard diode-array spectrophotometer (model 8452A) using quartz cells (1-cm path length). Optical band gaps were calculated from the onset of the UV-visible absorption band. For solid-state measurements, polymer solutions in chloroform were cast on quartz plates. Fluorescence spectra were measured using a Varian Eclipse spectrofluorimeter. For fluorescence analyses in solution, the polymer concentration was about 10⁻⁶ M. The fluorescence quantum yield (φ_{F}) for PCVBN was determined in argon-saturated chloroform solutions at 298° K using 9,10-diphenylanthracene (Aldrich) in cyclohexane as the standard (φ_{F}=0.90). The fluorescence quantum yield for PCV, PCVP and PCVDPATA was determined against PQC10 (φ_{F}=0.11) in chloroform¹³, while 1,3,5,7,8-pentamethyl-2,6-diethylpyrromethane.BF₂ (+F=0.83) in ethanol¹⁴ was used for PCCVP and PCVDPAP. For solid-state fluorescence analyses, polymer solutions were cast on a triangular quartz cell and placed at 45° with respect to the incident beam. All fluorescence excitation spectra were found to be equivalent to their respective absorption spectra.

Materials: Chloroform (spectrograde) was purchased from Aldrich and used as received. 2,5-bis(diphenylamino)terephthaldicarboxaldehyde, [4-(2-ethylhexyloxy)-phenyl]-bis-(4′-formylphenyl)amine, 2,5-dioctyloxy-1,4-diformylbenzene, 6,6′-dibromo-2,2′-bis(2″-ethylhexyloxy)-1,1′-binaphthyl and 3-hexyl-2,5-bis(methylphosphonate)thiophene were synthesized as previously described in literature.^{15,16,17,18,19}

The present invention is illustrated in further detail by the following non-limiting examples.

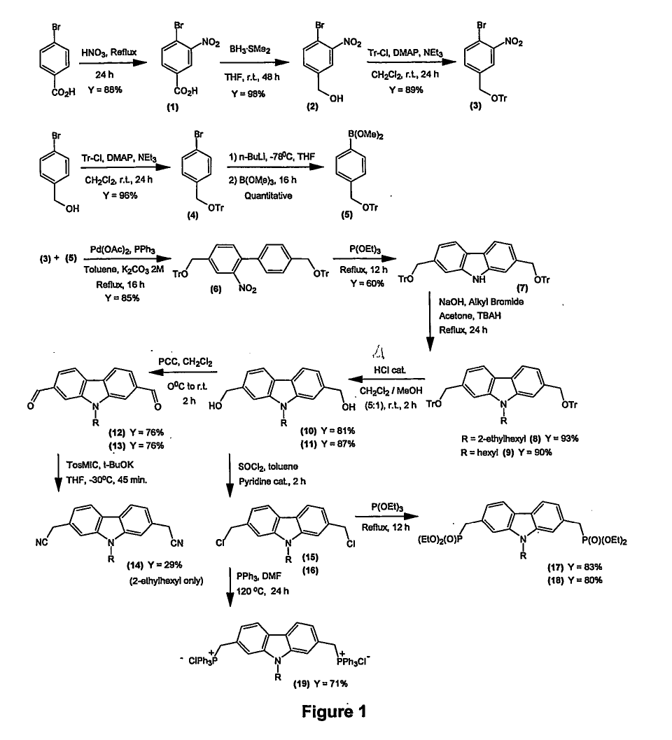

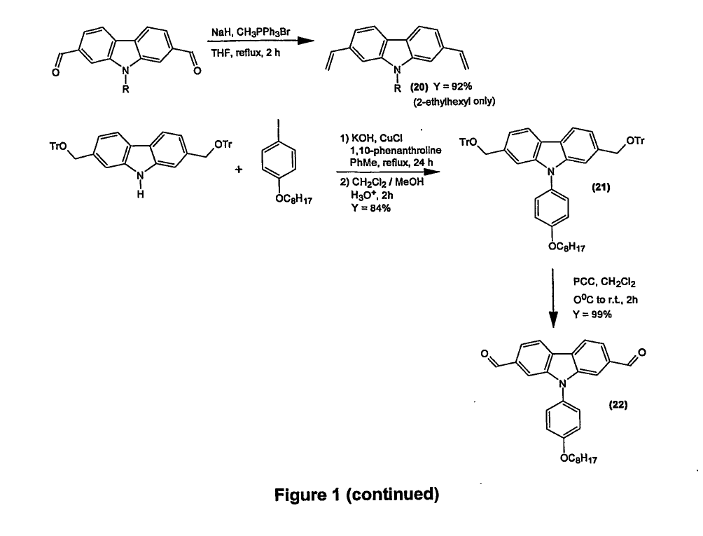

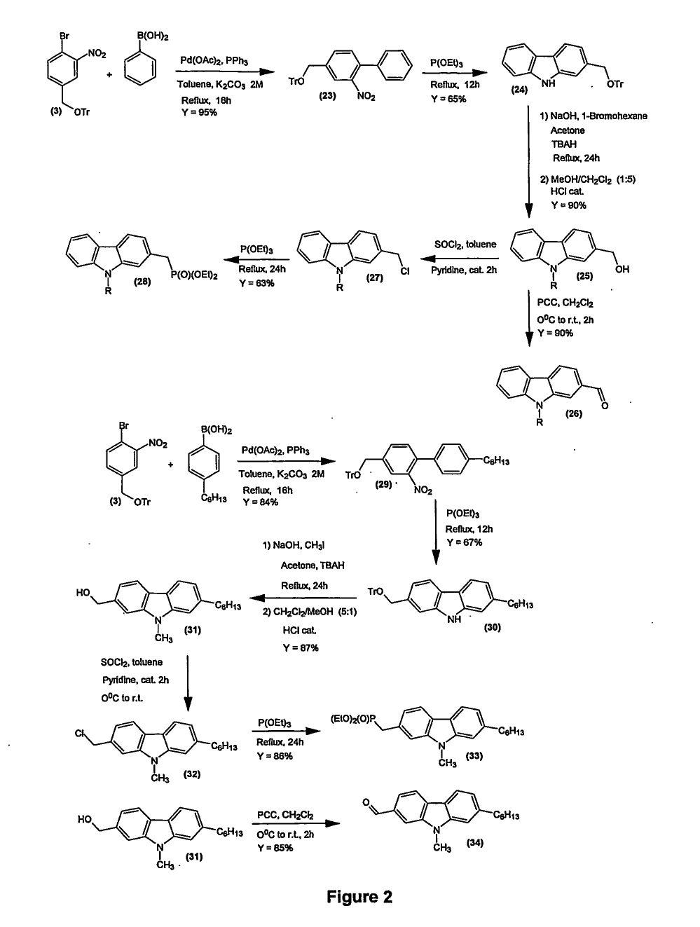

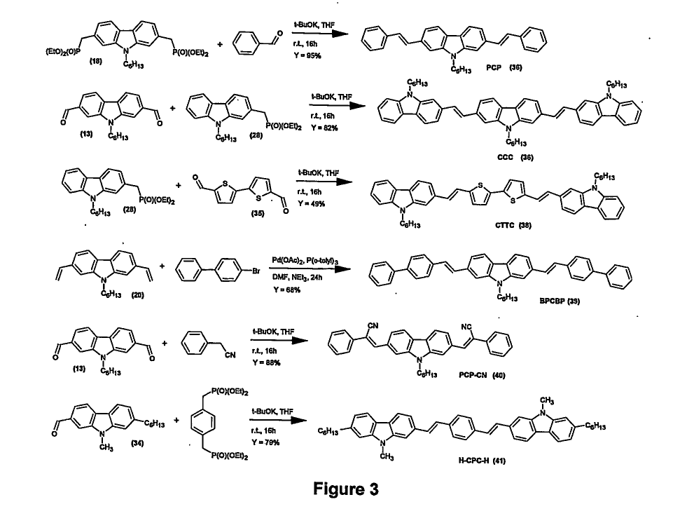

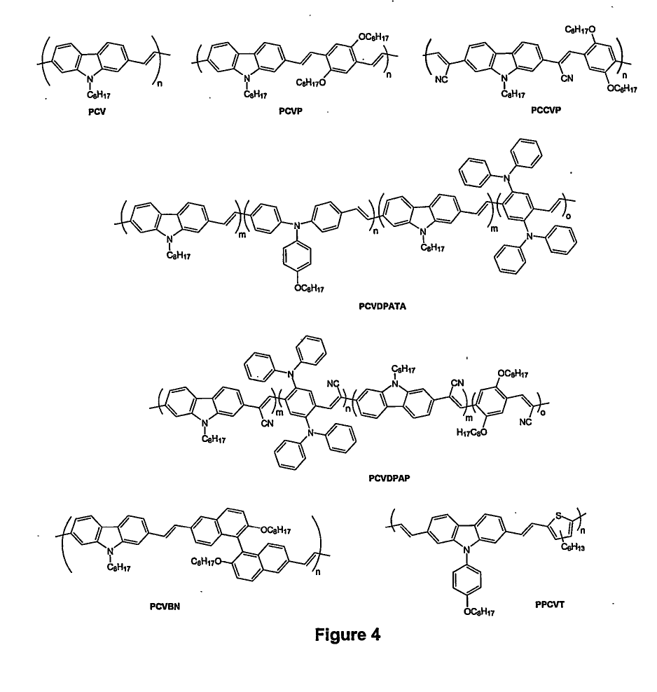

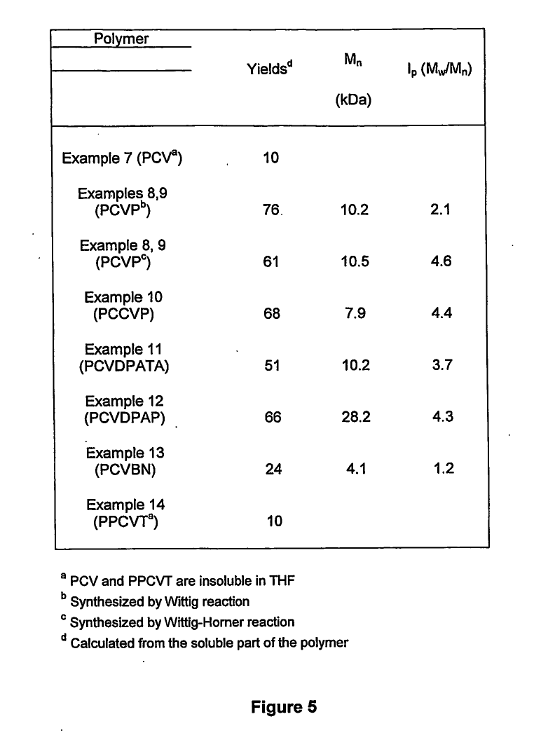

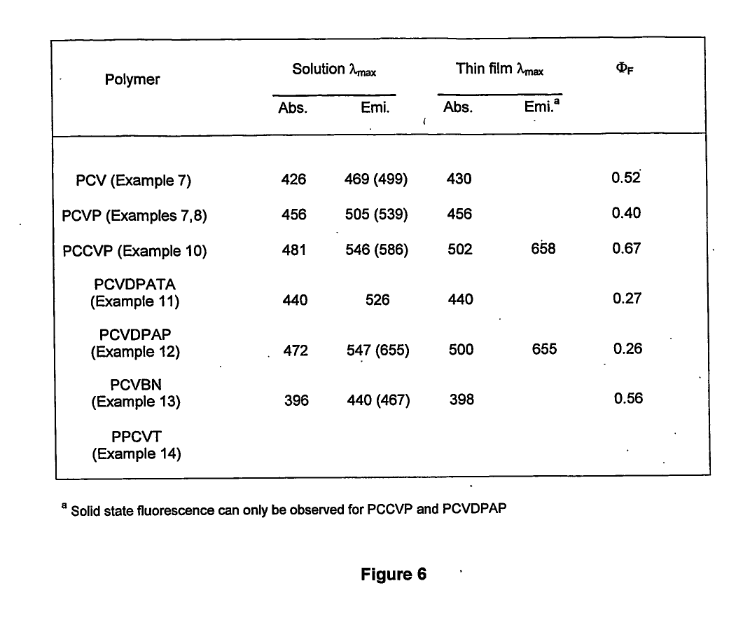

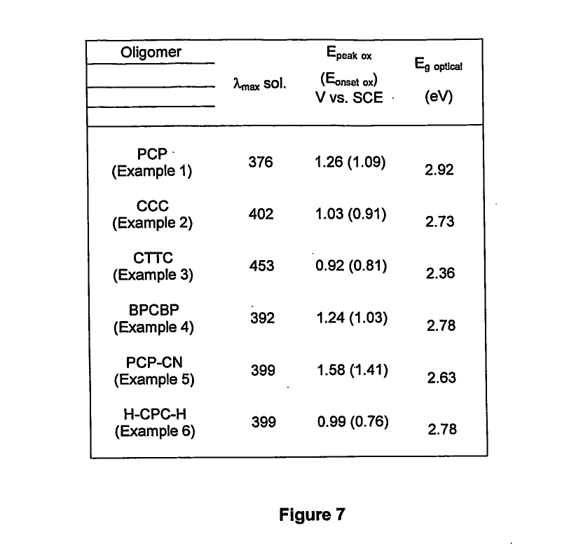

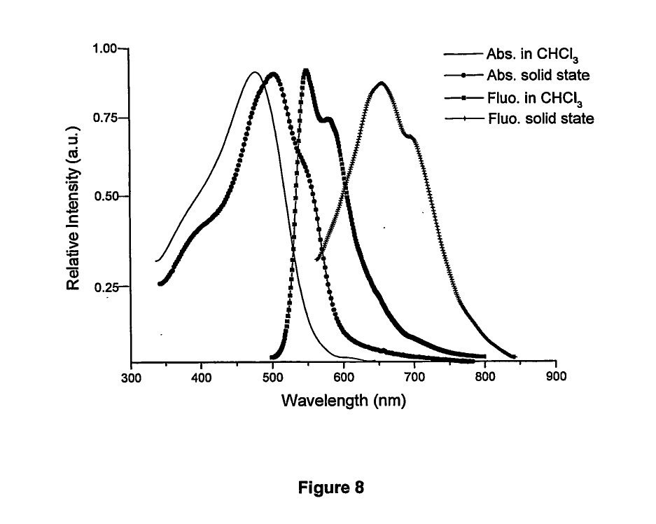

The following is a detailed description of precursors and reagents as well as the reaction schemes used to prepare the oligomers and polymers of the present invention. The number in between parenthesis refers to compounds in the reaction schemes depicted in FIGS. 1-3.

4-bromo-3-nitrobenzoic acid (1): In a 1 L flask, 4-bromobenzoic acid (50.0 g, 0.25 mol, Aldrich Co.), nitric acid (450 mL) and fuming nitric acid (100 mL) were mixed and refluxed for 24 h. The mixture was cooled at 0° C. and the white precipitate filtered through a Buchner funnel, washed thoroughly with water and dried under reduced pressure to provide 53.9 g of the title product as a white solid. M.P.: 202-204° C. (Yield: 88%). ¹H NMR (300 MHz, Acetone-d₆, ppm): 11.37 (s, 1H); 8.47 (d, 1H, J=1.9 Hz); 8.16 (dd, 1H, J=6.6 and 1.6 Hz); 8.04 (d, 1H, J=8.3 Hz). ¹³C NMR (75 MHz, Acetone-d₆, ppm): 206.35; 165.07; 136.29; 134.55; 132.26; 126.90; 119.08.

4-bromo-3-nitrobenzyl alcohol (2): To a solution of compound 1 (45.0 g, 0.18 mol) in 700 mL of anhydrous THF was slowly added borane-dimethylsulfide complex (19.4 mL, 0.19 mol, 10.0M in dimethylsulfide, Aldrich Co.) at room temperature. The mixture was stirred for 48 h under argon at room temperature and then quenched with 250 mL of distillated water. Diethyl ether (500 mL) was added and the organic layer was washed three times with water (250 mL) followed by brine (250 mL). The combined organic fractions were dried over magnesium sulfate and the solvent was removed under reduced pressure to provide 41.3 g of the title product as a yellow solid. M.P.: 61-62° C. (Yield: 98%). ¹H NMR (400 MHz, CDCl₃, ppm): 7.81 (s, 1H); 7.67 (d, 1H, J=8.3 Hz); 7.38 (d, 1H, J=8.5 Hz); 4.71 (s, 2H); 2.61 (s, 1H). ¹³C NMR (100 MHz, CDCl₃, ppm): 149.88; 142.16; 135.03; 131.17; 123.45; 112.88; 63.19.

Triphenylmethyl-(4-bromo-3-nitrobenzyl)ether (3)²⁰: In a 1 L flask, compound 2 (42.0 g, 0.18 mol), trityl chloride (56.0 g, 0.20 mol, Aldrich Co.), dimethylaminopyridine (0.89 g, 7.30 mmol, Aldrich Co.), triethylamine (46 mL, Aldrich Co.) and dichloromethane (400 mL) were mixed and stirred for 24 h. Distillated water (250 mL) was added and the organic layer was washed two times with a saturated NH₄Cl solution followed by water. The combined organic fractions were dried over magnesium sulfate and the solvent was removed under reduced pressure. The crude material was recrystallized in ethanol to provide 76.4 g of the title product as a yellow crystalline solid. M.P.: 148-150° C. (Yield: 89%). ¹H NMR (400 MHz, CDCl₃, ppm): 7.83 (s, 1H); 7.69 (d, 1H, J=8.3 Hz); 7.53 (d, 1H, J=7.2 Hz); 7.34 (m, 15H); 4.29 (s, 2H). ¹³C NMR (100 MHz, CDCl₃, ppm): 149.98; 143.57; 140.62; 134.81; 131.46; 128.64; 128.14; 127.45; 123.79; 112.55; 87.71; 64.33.

Triphenylmethyl-(4-bromobenzyl)ether (4)¹⁹: In a 1 L flask, 4-bromobenzyl alcohol (50.0 g, 0.27 mol, Aldrich Co.), trityl chloride (82.0 g, 0.29 mol, Aldrich Co.), dimethylaminopyridine (1.31 g, 10.6 mmol, Aldrich Co.), triethylamine (67 mL, Aldrich Co.) and dichloromethane (550 mL) were mixed and stirred for 24 h. Distilled water (300 mL) was added and the organic layer was washed two times with a saturated NH₄Cl solution followed by water. The combined organic fractions were dried over magnesium sulfate and the solvent was removed under reduced pressure. The crude material was recrystallized in ethanol to provide 111 g of the title product as a white crystalline solid. M.P.: 149-150° C. (Yield: 96%). ¹H NMR (300 MHz, CDCl₃, ppm): 7.59 (d, 2H, J=7.4 Hz); 7.53 (d, 2H, J=8.4 Hz); 7.35 (m, 15H); 4.23 (s, 2H). ¹³C NMR (75 MHz, CDCl₃, ppm): 144.06; 138.24; 131.46; 128.78 (2C); 128.03; 127.25; 121.00; 87.27; 65.26.

Triphenylmethyl-(4-(dimethoxyborane)benzyl)ether (5): To a solution of compound 4 (50.0 g, 0.12 mol) in anhydrous THF (500 mL) was added dropwise n-butyllithium (51.7 mL, 0.13 mol, 2.5 M in hexanes, Aldrich Co.) at −78° C. under argon. The mixture was stirred 2 h at −78° C. during which the solution turned pink followed by the formation of a white precipitate. Trimethylborate (26.4 mL, 0.24 mol, Aldrich Co.) was then added dropwise and the solution turned clear. The mixture was stirred at −78° C. for an additional hour followed by 16 h at room temperature. The solution was then quenched with an aqueous saturated NaHCO₃ solution (550 mL). Diethyl ether (500 mL) was added and the organic layer was washed three times with water (200 mL) followed by brine (200 mL). The combined organic fraction was dried over magnesium sulfate and the solvent was removed under reduced pressure to give a colorless oil that was used in the next step without further purification.

4,4′-bis(trityloxymethyl)-2-nitrobiphenyl (6): In a 250 mL flask, compound 3 (42.4 g, 89.4 mmol), compound 5 (39.7 g, 94.0 mmol), toluene (200 mL) and aqueous K₂CO₃ (2 M, 75 mL) were mixed. The resulting solution was degassed with a vigorous flow of argon for 1 h. Palladium (II) acetate (0.42 g, 1.88 mmol, Aldrich Co.) and triphenylphosphine (1.98 g, 7.52 mmol, Aldrich Co.) were then added and the mixture was refluxed for 16 h under argon. The mixture was cooled at room temperature and the white precipitate was filtered through a Buchner funnel. The resulting solid was washed thoroughly with water followed by methanol and dried under reduced pressure to provide 65.8 g of the title product as a white solid. M.P.: 250-251° C. (Yield: 85%). ¹H NMR (300 MHz, CDCl₃, ppm): 7.87 (s, 1H); 7.58 (m, 14H); 7.38 (m, 22H); 4.36 (s, 2H); 4.30 (s, 2H). ¹³C NMR (75 MHz, CDCl₃, ppm): 149.32; 144.11; 143.73; 140.08; 139.37; 136.06; 134.82; 131.87; 130.53; 128.80; 128.71; 128.10; 127.97; 127.90; 127.37; 127.21; 127.16; 122.36; 87.58; 87.15; 65.40; 64.67.

2,7-bis(trityloxymethyl)carbazole (7): In a 500 mL flask, compound 6 (40.0 g, 54.2 mmol) and triethylphosphite (250 mL) were mixed and refluxed under argon for 12 h. The mixture was cooled at 0° C. and the precipitate was filtered through a Buchner funnel. The solid was washed thoroughly with methanol and dried under reduced pressure to provide 23.0 g of the title product as a white solid. M.P.: 240° C. (dec.) (Yield: 60%). ¹H NMR (400 MHz, THF-d₈, ppm): 10.24 (s, 1H); 7.94 (d, 2H, J=8.0 Hz); 7.53 (m, 14H); 7.28 (m, 12H); 7.20 (m, 6H); 7.08 (dd, 2H, J=8.0 and 1.4 Hz); 4.30 (s, 4H). The ¹³C NMR experiment could be performed on this compound due to its very low solubility in common deuterated solvents.

N-(2-ethylhexyl)-2,7-bis(trityloxymethyl)carbazole (8)₉: A 250 mL flask was charged with compound 7 (20.0 g, 28.4 mmol), sodium hydroxide (2.28 g, 56.8 mmol), tetrabutylammonium hydrogensulfate (0.48 g, 1.42 mmol), 2-ethylhexylbromide (11.0 g, 57.0 mmol, Aldrich Co.) and anhydrous acetone (140 mL). The resulting mixture was refluxed under argon for 24 h and then cooled at room temperature. Water (300 mL) was then added under vigorous stirring and the white precipitate formed was collected by filtration. The solid was dissolved in a small amount of acetone and poured into methanol at 0° C. The precipitate was filtered and rinsed thoroughly with methanol to provide 21.6 g of the title product as a white solid. M.P.: 180-182° C. (Yield: 93%). ¹H NMR (300 MHz, CDCl₃, ppm): 8.15 (d, 2H, J=8.0 Hz); 7.74 (d, 12H, J=7.6 Hz); 7.68 (s, 2H); 7.46 (m, 12H); 7.39 (m, 6H); 7.31 (d, 2H, J=8.0 Hz); 4.55 (s, 4H); 4.34 (m, 2H); 2.30 (m, 1H); 1.47 (m, 8H); 1.11 (t, 3H, J=7.2 Hz); 0.94 (t, 3H, J=6.8 Hz). ¹³C NMR (75 MHz, CDCl₃, ppm): 144.46; 141.55; 136.86; 128.97; 128.02; 127.20; 122.03; 120.02; 118.12; 107.57; 87.25; 66.66; 39.60; 31.21; 28.92; 28.56; 24.56; 23.24; 14.17; 11.14.

N-hexyl-2,7-bis(trityloxymethyl)carbazole (9)₉: This product was obtained (via compound 7) following the same procedure as used for the synthesis of compound 8 using 1-bromohexane instead of 2-ethylhexylbromide to provide the title product as a white solid. M.P.: 183-184° C. (Yield: 90%). ¹H NMR (300 MHz, CDCl₃, ppm): 8.13 (d, 2H, J=8.0 Hz); 7.71 (d, 12H, J=7.6 Hz); 7.56 (s, 2H); 7.44 (m, 12H); 7.36 (m, 8H); 4.52 (s, 4H); 4.39 (t, 2H, J=7.0 Hz); 2.00 (m, 2H); 1.48 (m, 6H); 0.96 (t, 3H, J=6.8 Hz). ¹³C NMR (75 MHz, CDCl₃, ppm): 144.44; 141.00; 136.84; 128.96; 128.01; 127.18; 122.07; 120.11; 118.21; 107.35; 87.26; 66.72; 43.23; 31.76; 29.11; 27.18; 22.72; 14.18.

N-(2-ethylhexyl)-2,7-bis(hydroxymethyl)carbazole (10): A 500 mL flask was charged with compound 8 (20.0 g, 24.6 mmol), dichloromethane (500 mL), methanol (100 mL) and concentrated HCl (2 mL). The resulting mixture was stirred for 2 h, which was followed by the addition of saturated aqueous NaHCO₃ (200 mL). The aqueous layer was removed and the organic layer was washed three times with distilled water (200 mL). The combined organic layer was dried over magnesium sulfate and the solvent was removed under reduced pressure. The resulting solid was recrystallized twice in toluene to provide 6.44 g of the title product as a white solid. M.P.: 119-120° C. (Yield: 81%). ¹H NMR (300 MHz, Acetone-d₆, ppm): 8.04 (d, 2H, J=8.0 Hz); 7.55 (s, 2H); 7.18 (d, 2H, J=7.9); 4.83 (s, 2H); 4.82 (s, 4H); 4.28 (m, 2H); 2.13 (m, 1H); 1.40 (m, 6H); 1.25 (m, 2H); 0.92 (t, 3H, J=7.4 Hz); 0.84 (t, 3H, J=7.2 Hz). ¹³C NMR (75 MHz, Acetone-d₆, ppm): 142.14; 140.98; 122.39; 120.34; 118.45; 107.93; 65.32; 47.59; 39.89; 31.43; 29.18; 24.83; 23.58; 14.11; 11.10.

N-hexyl-2,7-bis(hydroxymethyl)carbazole (11): This product was obtained (via compound 9) following the same procedure as used for the synthesis of compound 10 to provide the title product as a white solid. M.P.: 96-97° C. (Yield: 87%). ¹H NMR (400 MHz, Acetone-d₆, ppm): 8.03 (d, 2H, J=8.0 Hz); 7.55 (s, 2H); 7.18 (d, 2H, J=7.9 Hz); 4.83 (d, 4H, J=5.8 Hz); 4.36 (t, 2H, J=7.3 Hz); 4.31 (t, 2H, J=5.8 Hz); 1.85 (m, 2H); 1.34 (m, 6H); 0.85 (t, 3H, J=7.1 Hz). ¹³C NMR (100 MHz, Acetone-d₆, ppm): 141.12; 140.43; 121.86; 119.85; 117.93; 107.14; 64.80; 42.69; 31.69; 29.02; 26.81; 22.59; 13.63.

N-(2-ethylhexyl)-2,7-bis(formyl)carbazole (12)²¹: In a 250 mL flask, compound 10 (5.00 g, 14.8 mmol), pyridinium chlorochromate (PCC) (12.8 g, 59.3 mmol, Aldrich Co.), dry molecular sieves 4A (2.50 g, Aldrich Co.) and silica gel (2.50 g) were added to dichloromethane (150 mL) at 0° C. The resulting mixture was stirred 2 h at room temperature and then filtered over silica gel (dichloromethane as eluent) to provide the title product as a bright yellow solid. M.P.: 120-121° C. (Yield: 76%). ¹H NMR (300 MHz, CDCl₃, ppm): 10.14 (s, 2H); 8.20 (d, 2H, J=8.01 Hz); 7.90 (s, 2H); 7.74 (d, 2H, J=8.04 Hz); 4.20 (d, 2H, J=7.6 Hz); 2.06 (s, 1H); 1.29 (m, 8H); 0.89 (t, 3H, J=7.4 Hz); 0.82 (t, 3H, J=6.8 Hz). ¹³C NMR (75 MHz, CDCl₃, ppm): 192.24; 142.13; 135.16; 126.75; 121.70; 121.18; 110.62; 47.84; 39.38; 30.81; 28.54; 24.34; 22.97; 13.94; 10.84.

N-hexyl-2,7-bis(formyl)carbazole (13)²¹: This product was obtained (via compound 11) following the same procedure as used for the synthesis of compound 12 to provide the title product as a bright yellow solid. M.P.: 98-99° C. (Yield: 76%). ¹H NMR (400 MHz, CDCl₃, ppm): 10.16 (s, 2H); 8.22 (d, 2H, J=8.4 Hz); 7.95 (s, 2H); 7.75 (dd, 2H, J=8.0 and 0.9 Hz); 4.36 (t, 2H, J=7.4 Hz); 1.88 (m, 2H); 1.34 (m, 6H); 0.84 (t, 3H, J=7.0 Hz). ¹³C NMR (100 MHz, CDCl₃, ppm): 192.56; 141.87; 135.34; 127.00; 121.96; 121.55; 110.44; 43.77; 31.68; 29.29; 27.08; 22.72; 14.18.

N-(2-ethylhexyl)-2,7-bis(acetonitrile)carbazole (14)₂₂: To a solution of potassium tert-butoxide (7.23 g, 67.1 mmol, Aldrich Co.) in THF (150 mL) was slowly added under argon a solution of tosylmethyl isocyanide (6.26 g, 32.0 mmol, Aldrich Co.) in anhydrous THF (50 mL). The resulting mixture was cooled at −30° C. and a solution containing compound 12 (5.00 g, 14.9 mmol) in anhydrous THF (50 mL) was slowly added. The mixture was stirred at −30° C. for 45 minutes followed by the addition of MeOH (200 mL). The solution was heated at 80° C. for 15 minutes and cooled at room temperature. The solvent was removed under reduced pressure and 10 mL of glacial acetic acid was added to the resulting dark solid. Water (100 mL) was added and the solid washed three times with dichloromethane. The combined organic layer was dried over magnesium sulfate and the solvent was removed under reduced pressure. The crude dark red viscous oil was purified by column chromatography (silica gel, 30% ethyl acetate in hexanes as eluent) to provide the title product as a slightly yellow solid. M.P.: 79-80° C. (Yield: 29%). ¹H NMR (300 MHz, CDCl₃, ppm): 8.01 (d, 2H, J=8.0 Hz); 7.32 (s, 2H); 7.13 (d, 2H, J=8.0 Hz); 4.04 (m, 2H); 3.95 (s, 4H); 2.02 (m, 1H); 1.33 (m, 8H); 0.80 (m, 6H). ¹³C NMR (75 MHz, CDCl₃, ppm): 141.50; 127.72; 122.03; 120.99; 119.02; 118.35; 108.55; 47.37; 39.31; 30.92; 28.69; 24.38; 24.26; 23.06; 14.03; 10.94.

N-(2-ethylhexyl)-2,7-bis(chloromethyl)carbazole (15): To a solution of compound 10 (5.00 g, 14.8 mmol) in dry toluene (140 mL) containing a few drops of pyridine was slowly added thionyl chloride (6.48 mL, 88.9 mmol, Aldrich Co.) at 0° C. The mixture was stirred at 0° C. for 1 h and at room temperature for 2 h. The excess thionyl chloride and toluene were removed under reduced pressure. The crude product was purified by column chromatography (silica gel, 10% ethyl acetate in hexanes as eluent). The yellow oil obtained was decolorized using activated carbon to provide 3.82 g of the title product as a slightly yellow solid. (Yield: ˜85%). (The final product contained 5-10% of unknown impurities and was used as is).

N-hexyl-2,7-bis(chloromethyl)carbazole (16): This product was obtained (via compound 11) following the same procedure as used for the synthesis of compound 15 to provide the title product as a slightly yellow solid. (Yield: ˜82%). (The final product contained 5-10% of unknown impurities and was used as is).

N-(2-ethylhexyl)-2,7-bis(methylphosphonate)carbazole (17): In a 100 mL flask, compound 15 (3.80 g, 12.5 mmol) and triethylphosphite (50 mL) were mixed and heated to reflux under argon for 0.24 h. The excess triethylphosphite was removed under reduced pressure and the crude product was purified by column chromatography (silica gel, 50% acetone in hexanes as eluent) to provide 4.86 g of the title product as a yellow waxy solid. M.P.: 70-71° C. (Yield: 83%). ¹H NMR (300 MHz, CDCl₃, ppm): 7.89 (d, 2H, J=8.0 Hz); 7.26 (s, 2H); 7.06 (d, 2H, J=7.9 Hz); 4.06 (m, 2H); 3.92 (m, 8H); 3.28 (d, 4H, J=21.3 Hz); 1.99 (m, 1H); 1.27 (m, 8H); 1.15 (t, 12H, J=7.0 Hz); 0.79 (m, 6H). ¹³C NMR (75 MHz, CDCl₃, ppm): 141.33; 128.92; 128.80; 121.48; 120.83; 120.76; 120.06; 110.19; 110.10; 62.05; 61.97; 47.45; 39.20; 35.37; 33.54; 30.94; 28.76; 24.31; 22.96; 16.38; 16.31; 13.94; 10.90.

N-hexyl-2,7-bis(methylphosphonate)carbazole (18): This product was obtained (via compound 16) following the same procedure as used for the synthesis of compound 17. The crude product was purified by column chromatography (silica gel, 50% acetone in hexanes as eluent) to provide the title product as a white solid. M.P.: 117-119° C. (Yield: 80%). ¹H NMR (400 MHz, CDCl₃, ppm): 7.97 (d, 2H, J=7.9 Hz); 7.35 (s, 2H); 7.12 (d, J=7.9 Hz); 4.26 (t, 2H, J=7.3 Hz); 3.99 (m, 8H); 3.36 (d, 4H, J=21.5 Hz); 1.84 (m, 2H); 1.34 (m, 6H); 1.23 (t, 12H, J=7.0 Hz); 0.85 (t, 3H, J=6.9 Hz). ¹³C NMR (100 MHz, CDCl₃, ppm): 141.11; 129.16; 126.06; 121.76; 121.06; 121.00; 120.40; 110.15; 110.07; 62.37; 62.30; 43.36; 35.40; 33.99; 31.86; 29.17; 27.16; 22.75; 16.65; 16.59; 14.24.

N-(2-ethylhexyl)-2,7-bis(methyltriphenylphosphonium chloride)carbazole (19): In a 100 mL flask, compound 15 (3.00 g, 7.98 mmol), triphenylphosphine (5.23 g, 19.9 mmol) and anhydrous DMF (80 mL) were stirred at 120° C. under argon for 24 h. The mixture was cooled at room temperature and poured in 300 mL of cold diethyl ether under vigorous stirring. The slightly yellow precipitate was filtered and washed thoroughly with diethyl ether. The solid was dissolved in water and extracted five times with dichloromethane. The combined organic fractions were dried over magnesium sulfate and the solvent was removed under reduced pressure to provide 5.13 g of the title product as a slightly yellow solid. M.P. >260° C. (Yield: 71%). ¹H NMR (400 MHz, CDCl₃, ppm): 7.91 (m, 10H); 7.72 (m, 22H); 7.21 (s, 2H); 6.83 (m, 2H); 5.20 (d, 4H, J=14.7 Hz); 3.80 (m, 2H); 1.44 (m, 1H); 0.95 (m, 8H); 0.78 (t, 3H, J=6.7 Hz); 0.71 (t, 3H, J=7.3 Hz). ¹³C NMR (100 MHz, CDCl₃, ppm): 141.37; 135.30; 134.34; 130.26; 125.38; 122.42; 120.83; 118.69; 117.80; 111.93; 53.87; 38.38; 30.64; 30.47; 29.99; 29.69; 28.67; 24.29; 22.90.

N-(2-ethylhexyl)-2,7-divinylcarbazole (20): In a 100 mL flask, compound 12 (2.00 g, 5.96 mmol), sodium hydride (0.36 mg, 14.9 mmol, Aldrich Co.), methyl triphenylphosphonium bromide (5.11 g, 14.3 mmol, Aldrich Co.) and anhydrous THF (60 mL) were heated to reflux under argon for 2 h. The resulting solution was cooled at room temperature and methanol (50 mL) was slowly added followed by water (50 mL). The aqueous layer was washed three times with dichloromethane (100 mL) and the combined organic fractions were washed with brine followed by water. The organic layer was dried over magnesium sulfate and the solvent was removed under reduced pressure. The crude product was purified by column chromatography (5% ethyl acetate in hexanes as eluent) to provide 1.80 g of the title product as a pale yellow solid. M.P.: 59-60° C. (Yield: 92%). ¹H NMR (400 MHz, CDCl₃, ppm): 8.03 (d, 2H, J=7.9 Hz); 7.39 (m, 4H); 6.98 (dd, 2H, J=8.2 Hz and 6.6 Hz); 5.95 (d, 2H, J=0.9 Hz); 5.90 (d, 2H, J=0.9 Hz); 4.13 (m, 2H); 2.11 (m, 1H); 1.40 (m, 8H); 0.98 (m, 6H). ¹³C NMR (100 MHz, CDCl₃, ppm): 142.06; 138.17; 135.57; 122.81; 120.50; 117.66; 113.31; 107.15; 47.38; 39.62; 31.20; 29.02; 24.73; 23.34; 14.37; 11.23.

N-(4-octyloxyphenyl)-2,7-bis(hydroxymethyl)carbazole (21): In a 50 mL flask, compound 7 (6.00 g, 8.52 mmol), 4-octyloxy-1-iodobenzene (3.40 g, 10.2 mmol), potassium hydroxide (3.20 g, 57.1 mmol), copper (I) chloride (67 mg, 0.68 mmol, Aldrich Co.), 1,10-phenanthroline (67 mg, 0.37 mmol) and toluene (25 mL) were mixed and refluxed for 24 h. The mixture was cooled at room temperature and poured into water. The aqueous layer was extracted three times with dichloromethane and the combined organic layers were dried over magnesium sulfate. The solvent was removed under reduced pressure and the resulting crude product was dissolved in a mixture of dichloromethane (250 mL) and methanol (75 mL) containing a few drops of concentrated HCl (1 mL). The resulting mixture were stirred for 2 h followed by the addition of saturated aqueous NaHCO₃ (100 mL). The aqueous layer was removed and the organic layer was washed three times with distilled water (100 mL). The combined organic layers were dried over magnesium sulfate and the solvent was removed under reduced pressure. During evaporation, a white precipitate was formed which was subsequently separated from solution by filtration. This process was repeated until a precipitate was no longer formed. The combined precipitates were dried under reduced pressure to provide 2.89 g of the title product as a white solid (Yield: 94%). ¹H RMN (400 MHz, Acetone-d₆, ppm): 8.10 (d, 2H, J=8.0 Hz); 7.48 (d, 2H, J=8.9 Hz); 7.34 (s, 2H); 7.23 (m, 4H); 4.76 (d, 4H, J=5.9 Hz); 4.22 (t, 2H, J=5.8 Hz); 4.14 (t, 2H, J=6.5 Hz); 1.86 (m, 2H); 1.55 (m, 2H); 1.38 (m, 8H); 0.91 (t, 3H, J=7.0 Hz). ¹³C RMN (100 MHz, Acetone-d₆, ppm): 158.92; 142.04; 140.90; 130.17; 128.81; 122.12; 119.89; 118.80; 115.86; 107.69; 68.28; 64.53; 31.91; 29.42; 29.36; 26.14; 22.65; 13.69.

N-4-octyloxyphenyl)-2,7-bis(formyl)carbazole (22): In a 100 mL flask, compound 21 (1.50 g, 3.48 mmol), pyridinium chlorochromate (3.75 g, 17.4 mmol, Aldrich Co.), molecular sieves 4A (750 mg), silica gel (750 mg) and dichloromethane (35 mL) were mixed at room temperature. The resulting mixture was stirred at room temperature for 2 h and then filtered onto silica gel (dichloromethane as eluent) to provide the title product as a bright yellow solid. M.P.: (Yield: 99%). ¹H RMN (400 MHz, CDCl₃, ppm): 10.08 (s, 2H); 8.29 (d, 2H, J=8.1 Hz); 7.86 (s, 2H); 7.83 (dd, 4H, J=8.0 and 1.3 Hz); 7.41 (d, 2H, J=8.9 Hz); 7.14 (d, 2H, J=8.9 Hz); 4.08 (t, 2H, J=6.6 Hz); 1.87 (m, 2H); 1.53 (m, 2H); 1.36 (m, 8H); 0.91 (t, 3H, J=6.8 Hz). ¹³C RMN (100 MHz, CDCl₃, ppm): ¹³C RMN (100 MHz, CDCl₃, ppm): 192.46; 159.57; 143.00; 135.60; 128.78; 128.45; 121.95; 121.74; 121.12; 116.27; 112.32; 68.71; 32.06; 29.59; 29.50; 29.45; 26.30; 22.90; 14.36.

4-methyltrityloxy-2-nitrobiphenyl (23): In a 500 mL flask, compound 3 (55.0 g, 117 mmol), phenylboronic acid (15.0 g, 123 mmol, Aldrich Co.), toluene (180 mL) and aqueous K₂CO₃ 2 M (70 mL) were mixed. The resulting solution was degassed with a vigorous flow of argon for 1 h. Palladium (II) acetate (0.55 g, 2.46 mmol, Aldrich Co.) and triphenylphosphine (2.58 g, 9.84 mmol, Aldrich Co.) were then added and the mixture was refluxed for 16 h under argon. The mixture was cooled at room temperature and water (200 mL) was added. The aqueous layer was washed three times with toluene (100 mL) and the combined organic fractions were dried with magnesium sulfate. The residue was filtered and the filtrate was decolorized by heating in presence of activated carbon followed by filtration on Celite®. The solvent was removed under reduced pressure and the crude product was purified by precipitation in ethanol to provide 51.9 g of the title product as a white solid. M.P.: 113-115° C. (Yield: 95%). ¹H NMR (400 MHz, CDCl₃, ppm): 7.88 (m, 1H); 7.63 (m, 1H); 7.58 (m, 3H); 7.56 (m, 3H); 7.44 (m, 4H); 7.38 (m, 8H); 7.31 (m, 3H); 4.36 (s, 2H). ¹³C NMR (100 MHz, CDCl₃, ppm): 149.44; 143.90; 140.35; 137.58; 135.17; 132.05; 130.76; 128.94; 128.88; 128.42; 128.30; 128.19; 127.57; 122.55; 87.75; 64.84.

2-methyltrityloxycarbazole (24): In a 500 mL flask, compound 23 (51.5 g, 110 mmol) and triethylphosphite (275 mL) were mixed and refluxed under argon for 12 h. The mixture was cooled at room temperature and excess triethylphosphite was removed under reduced pressure. Methanol (250 mL) was added and the precipitate was filtered through a Buchner funnel. The white precipitate was recrystallized in an ethyl acetate/hexanes mixture to provide 31.0 g of the title product as a white solid. M.P.: 228-230° C. (Yield: 65%). ¹H NMR (300 MHz, CDCl₃, ppm): 10.34 (s, 1H); 8.09 (m, 2H); 7.65 (m, 1H); 7.60 (m, 3H); 7.58 (m, 3H); 7.52 (m, 1H); 7.37 (m, 7H); 7.29 (m, 3H); 7.17 (m, 2H); 4.34 (s, 2H). ¹³C NMR (75 MHz, CDCl₃, ppm): 144.58; 140.57; 140.51; 136.94; 128.86; 128.07; 127.29; 125.60; 123.22; 122.50; 120.13; 120.06; 119.02; 118.38; 111.04; 109.59; 87.18; 86.45.

N-hexyl-2-hydroxymethylcarbazole (25): A 500 mL flask was charged with compound 24 (20.0 g, 45.9 mmol), sodium hydroxide (3.67 g, 91.8 mmol), tetrabutylamonium hydrogensulfate (0.78 g, 2.29 mmol), 1-bromohexane (15.2 g, 91.8 mmol, Aldrich Co.) and anhydrous acetone (230 mL). The resulting mixture was refluxed under argon for 24 h and then poured into 250 mL of distillated water. The aqueous layer was extracted three times with diethyl ether (100 mL). The combined organic fractions was dried over magnesium sulfate and the solvent was removed under reduced pressure to give an orange oil. The crude product was dissolved in dichloromethane (500 mL) and methanol (100 mL). Concentrated hydrochloric acid (2 mL) was added and the mixture was stirred for 30 minutes at room temperature. Saturated aqueous NaHCO₃ (200 mL) was then added. The aqueous layer was removed and the organic layer was extracted three times with distilled water (100 mL). The combined organic layer were dried over magnesium sulfate and the solvent was removed under reduced pressure. The residue was purified by column chromatography (30% ethyl acetate in hexanes as eluent) to provide 11.7 g of the title product as a white solid. M.P.: 54-55° C. (Yield: 90%). ¹H NMR (300 MHz, CDCl₃, ppm): 8.09 (t, 2H, J=8.5 Hz); 7.43 (m, 3H); 7.23 (m, 2H); 4.89 (s, 2H); 4.28 (t, 2H, J=7.4 Hz); 1.93 (s, 1H); 1.87 (m, 2H); 1.36 (m, 6H); 0.89 (t, 3H, J=6.9 Hz). ¹³C NMR (75 MHz, Acetone-d₆, ppm): 141.54; 141.50; 141.29; 126.09; 123.56; 122.52; 120.79; 120.66; 119.45; 118.60; 109.70; 107.75; 65.47; 43.30; 32.25; 29.60; 27.43; 23.18; 14.29.

N-hexyl-2-formylcarbazole (26): In a 250 mL flask, compound 25 (2.00 g, 7.11 mmol), pyridinium chlorochromate (PCC) (3.06 g, 14.2 mmol, Aldrich Co.), dry molecular sieves 4 Å (1.20 g, Aldrich Co.) and silica gel (1.20 g) were added to dichloromethane (70 mL) at 0° C. The resulting mixture was stirred 2 h at room temperature and then filtered onto silica gel (dichloromethane as eluent) to provide 1.79 g of the title product as an orange oil (Yield: 90%). ¹H NMR (300 MHz, CDCl₃, ppm): 10.15 (s, 1H); 8.14 (m, 2H); 7.92 (s, 1H); 7.71 (d, 1H, J=8.0 Hz); 7.55 (t, 1H, J=7.3 Hz); 7.41 (d, 1H, J=8.3 Hz); 7.27 (t, 1H, J=7.4 Hz); 4.27 (t, 2H, J=7.4 Hz); 1.85 (m, 2H); 1.30 (m, 6H); 0.88 (t, 3H, J=6.5 Hz). ¹³C NMR (75 MHz, CDCl₃, ppm): 192.63; 142.21; 140.06; 133.85; 128.08; 127.60; 121.92; 121.45; 121.18; 120.52; 119.59; 109.64; 109.22; 43.26; 31.55; 29.00; 26.93; 22.56; 14.03.

N-hexyl-2-chloromethylcarbazole (27): To a solution of compound 25 (5.00 g, 14.8 mmol) in dry toluene (140 mL) containing a few drops of pyridine, was slowly added thionyl chloride (6.48 mL, 88.9 mmol, Aldrich Co.) at 0° C. The mixture was stirred at 0° C. for 1 h and at room temperature for 2 h. Excess thionyl chloride and toluene were removed under reduced pressure. The crude product was purified by column chromatography (silica gel, 10% ethyl acetate in hexanes as eluent). The yellow oil obtained was decolorized using activated carbon to provide 3.82 g of the title product as a slightly yellow solid (Yield: ˜87%). (The final product contained 5-10% of unknown impurities and was used as is).

N-hexyl-2-methylphosphonatecarbazole (28): In a 250 mL flask, compound 27 (10.0 g, 33.3 mmol) and triethylphosphite (125 mL) were mixed and heated to reflux under argon for 24 h. The solution was cooled to room temperature and excess triethylphosphite was removed under reduced pressure. The resulting orange solution was purified by column chromatography (40% acetone in hexanes as eluent) to provide 8.40 g of the title product as a yellow viscous oil (Yield: 63%). ¹H NMR (300 MHz, CDCl₃, ppm): 8.08 (d, 1H, J=7.8 Hz); 8.03 (d, 1H, J=8.0 Hz); 7.46 (m, 1H); 7.38 (m, 2H); 7.23 (m, 1H); 7.16 (m, 1H); 4.28 (t, 2H, J=6.5 Hz); 4.02 (m, 4H); 3.38 (d, 2H, J=21.5 Hz); 1.86 (m, 2H); 1.34 (m, 6H); 1.24 (t, 6H, J=7.3 Hz); 0.88 (t, 3H, J=7.0 Hz). ¹³C NMR (75 MHz, CDCl₃, ppm): 140.86; 129.21; 129.12; 129.12; 125.74; 122.85; 122.83; 121.98; 121.95; 121.01; 120.94; 120.52; 120.50; 120.44; 119.03; 110.13; 110.06; 108.93; 62.39; 62.33; 43.29; 35.40; 34.03; 31.85; 29.16; 27.18; 22.78; 16.68; 16.62; 14.26.

4-Hexyl-4′-trityloxymethyl-2′-nitro-1,1′-biphenyl (29): In a 500 mL flask, 4-hexylphenylboronic acid (21.36 g, 104 mmol), 4-bromo-3-nitro(trityloxymethyl)benzene (46.3 g, 98 mmol), toluene (250 mL) and an aqueous solution of potassium carbonate 2 M (100 mL) were mixed. The resulting solution was degassed with a vigorous argon flow for 1 h. Palladium acetate (0.47 g, 2.10 mmol, Aldrich Co.) and triphenylphosphine (2.17 g, 8.40 mmol, Aldrich Co.) were then added and the solution was refluxed for 16 h under argon atmosphere. The solution was cooled at room temperature and distilled water (150 mL) was added. The organic layer was separated, washed three times with distilled water and dried over magnesium sulfate. The solvent was removed providing a yellow viscous oil. The organic layer was separated and washed three times with distilled water. Hot methanol (300 mL) was then added. The resulting mixture was stirred while being cooled in an ice/water bath. The obtained yellow precipitate was collected by filtration and dried under reduced pressure for 24 h to provide 33.9 g of the title product as a white powder. M.P.: 86-87° C. (Yield=84%). ¹H NMR (400 MHz, CDCl₃, ppm): 7.84 (s, 1H); 7.61 (d, 1H, J=0.8 Hz); 7.58 (m, 2H); 7.56 (m, 4H); 7.43 (d, 1H, J=7.9 Hz); 7.37 (m, 6H); 7.32 (m, 3H); 7.28 (m, 4H); 4.35 (s, 2H); 2.70 (t, 2H, J=8.2 Hz); 1.70 (m, 2H); 1.39 (m, 6H); 0.95 (t, 3H, J=7.2 Hz). ¹³C NMR (100 MHz, CDCl₃, ppm): 149.54; 143.91; 143.35; 140.01; 135.13; 134.69; 132.03; 130.65; 129.01; 128.88; 128.28; 128.03; 127.55; 122.48; 87.72; 64.85; 35.97; 31.99; 31.56; 29.33; 22.88; 14.40.

2-Hexyl-7-(trityloxymethyl)carbazole (30): In a 250 mL flask, compound 29 (32.0 g, 58.0 mmol) and triethylphosphite (150 mL, Aldrich Co.) were mixed. The resulting solution was refluxed for 16 h under argon atmosphere. Excess triethylphosphite was removed under reduced pressure. Ethanol (250 mL) was then added under vigorous stirring leading to a white precipitate. The solid was collected by filtration, rinsed thoroughly with methanol and dried under reduced pressure for 24 h to provide the title product as a white powder. M.P.: 155-156° C. (Yield: 67%). ¹H NMR (400 MHz, CDCl₃, ppm): 8.03 (t, 2H, J=6.5 Hz); 7.82, (s, 1H); 7.68 (m, 6H); 7.54 (s, 1H); 7.41 (m, 6H); 7.33 (m, 3H); 7.24 (m, 2H); 7.15 (dd, 1H, J=8.0 et 1.3 Hz); 4.43(s, 2H); 7.84 (t, 2H, J=8.0 Hz); 1.78 (m, 2H); 1.43 (m, 6H); 1.00 (t, 3H, J=7.0 Hz). ¹³C NMR (100 MHz, CDCl₃, ppm): 144.56; 141.29; 140.41; 140.01; 136.82; 129.09; 128.20; 127.36; 122.82; 121.48; 120.65; 120.16; 119.98; 118.83; 110.42; 109.25; 87.37; 66.61; 36.86; 32.27; 32.12; 29.40; 22.98; 14.48.

N-methyl-2-hexyl-7-(hydroxymethyl)carbazole (31): To a solution of compound 30 (19.33 g, 37.4 mmol) in anhydrous acetone (200 mL) were added sodium hydroxide (2.98 g, 74.5 mmol), tetrabutylamonium hydrogensulfate (0.39 g, 1.12 mmol) and iodomethane (10.6 g, 74.5 mmol). The resulting solution was refluxed for 4 h and then cooled to room temperature. Acetone was removed under reduced pressure and diethyl ether (250 mL) and distilled water (200 mL) were added. The organic layer was separated and washed two times with distilled water. The solvent was removed under reduced pressure and the resulting white solid was dissolved in a mixture of dichloromethane (400 mL) and methanol (100 mL) containing few drops of concentrated HCl. The resulting mixture was stirred for 1 h and a saturated aqueous sodium bicarbonate solution (250 mL) was added. The organic layer was separated, dried over magnesium sulfate and removed under reduced pressure. The crude was purified by column chromatography (silica gel, 30% ethyl acetate in hexanes as eluent) to provide 9.43 g of the title product as a white solid. M.P.: 90-91° C. (Yield: 87%). ¹H NMR (400 MHz, CDCl₃, ppm): 8.00 (d, 2H, J=7.9 Hz); 7.32 (s, 1H); 7.16 (m, 3H); 4.84 (s, 2H); 3.70 (s, 3H); 2.88 (t, 2H, J=7.7 Hz); 2.51 (s, 1H); 1.81 (m, 2H); 1.45 (m, 6H); 1.00 (t, 3H, J=7.1 Hz). ¹³C NMR (100 MHz, CDCl₃, ppm): 141.95; 141.52; 141.36; 138.45; 122.58; 120.76; 120.19; 120.14; 120.10; 118.14; 108.28; 107.10; 66.22; 37.12; 32.43; 32.13; 29.49; 29.08; 22.98; 14.47.

N-methyl-2-hexyl-7-chloromethylcarbazole (32): To a solution of compound 31 (2.00 g, 6.89 mmol) in anhydrous toluene (140 mL) at 0° C. was added thionyl chloride (1.64 g, 13.9 mmol). The resulting solution was stirred at 0° C. for 1 h and then for 2 h at room temperature. Excess thionyl chloride and toluene were removed under reduced pressure. The dark oil obtained was decolorized using activated carbon and was used as is without further purification.

N-methyl-2-hexyl-7-(methylphosphonate)carbazole (33): In a 25 mL flask were added compound 32 (2.10 g, 6.80 mmol) and triethylphosphite (10 mL). The resulting solution was refluxed for 24 h under argon atmosphere. Excess triethylphosphite was removed under reduced pressure. The crude product was purified by column chromatography (silica gel, 30% acetone in hexanes as eluent) to provide 2.42 g of the title product as an orange solid. M.P.: 61-62° C. (Yield: 86%). ¹H NMR (400 MHz, CDCl₃, ppm): 7.97 (m, 2H); 7.35 (m, 1H); 7.19 (s, 1H); 7.13 (dt, 1H, J=7.9 et 1.6 Hz); 7.08 (dd, 1H, J=7.9 et 1.4 Hz); 4.00 (m, 4H); 3.81 (s, 3H); 3.36 (d, 2H, J=21.4 Hz); 2.82 (t, 2H, J=7.7 Hz); 1.74 (m, 2H); 1.37 (m, 6H); 1.24 (t, 6H, J=7.1 Hz); 0.92 (t, 3H, J=7.0 Hz). ¹³C NMR (100 MHz, CDCl₃, ppm): 141.84; 141.59; 141.56; 141.35; 141.34; 129.58; 129.50; 122.06; 122.03; 120.95; 120.89; 120.71; 120.69; 120.12; 120.09; 120.08; 109.85; 109.77; 108.25; 62.43; 62.37; 37.03; 35.34; 33.97; 32.36; 32.04; 29.35; 29.24; 22.88; 16.67; 16.61; 14.37.

N-methyl-2-hexyl-7-(formyl)carbazole (34): To a solution of compound 31 (2.00 g, 6.89 mmol) in anhydrous dichloromethane (75 mL) at 0° C. were added pyridinium chlorochromate (PCC) (2.97 g, 13.9 mmol, Aldrich Co.), molecular sieves 4 Å (1.14 g) and silica gel (1.14 g). The resulting solution was stirred under argon atmosphere for 2 h at room temperature and then filtered onto silica gel (dichloromethane as eluent) to provide the title product as a bright yellow solid. M.P.: 55-56° C. (Yield: 85%). ¹H NMR (400 MHz, CDCl₃, ppm): 10.14 (s, 1H); 8.15 (d, 1H, J=8.0 Hz); 8.03 (d, 1H, J=8.0 Hz); 7.93(s, 1H); 7.72 (dd, 1H, J=8.0 et 1.4 Hz); 7.24 (s, 1H); 7.13 (dd, 1H, J=8.0 et 1.3 Hz); 3.90 (s, 3H); 2.83 (t, 2H, J=7.7 Hz); 1.73 (m, 2H); 1.34 (m, 6H); 0.90 (t, 3H, J=7.0 Hz). ¹³C NMR (100 MHz, CDCl₃, ppm): 192.90; 143.74; 143.43; 140.93; 133.58; 128.43; 121.62; 121.30; 121.00; 120.29; 120.00; 109.50; 108.63; 37.11; 32.18; 31.99; 29.43; 29.31; 22.85; 14.35.

5,5′-diformyl-2,2′-bithiophene (35): To a solution of 5,5′-dibromo-2,2′-bithiophene (2.00 g, 6.17 mmol, Aldrich Co.) in anhydrous THF (30 mL) was added dropwise n-butyllithium (5.43 mL, 13.6 mmol, 2.5 M in hexanes, Aldrich Co.) at −78° C. under argon. The mixture was stirred 30 min. at −78° C., warmed to room temperature and stirred for an additional 90 minutes. Anhydrous dimethylformamide (1.43 mL, 18.5 mmol, Aldrich Co.) was added dropwise and the solution was stirred at room temperature for another 2 h. An aqueous HCl solution (1 M, 10 mL) was slowly added followed by the addition of acetone (50 mL). The resulting mixture was poured into 150 mL of hexanes at 0° C. and the brown precipitate was filtered, washed with hexanes and dried under vacuum for 24 h to provide 1.05 g of the title product as a orange-brown solid. M.P.: 213-214° C. (Yield: 76%). ¹H NMR (400 MHz, DMSO-d₆, ppm): 9.89 (s, 2H); 8.00 (d, 2H, J=4.0 Hz); 7.73 (d, 2H, J=4.0 Hz). ¹³C NMR (100 MHz, DMSO-d₆, ppm): 185.00; 144.14; 144.03; 139.60; 128.57.

The following examples provide preferred embodiments of oligomers and polymers as contemplated by the present invention. Examples 1-6 are drawn to oligomers, whereas examples 7-14 are drawn to polymers.

### EXAMPLE 1

N-hexyl-2,7-bis(vinylenephenylene)carbazole (36) (PCP): To a solution of compound 18 (500 mg, 0.91 mmol) and benzaldehyde (240 mg, 2.27 mmol, Aldrich Co.) in anhydrous THF (20 mL) was slowly added potassium tert-butoxide (470 mg, 4.19 mmol, Aldrich Co.). The mixture was stirred at room temperature for 16 h and then poured into 300 mL of methanol. The yellow precipitate was filtered and washed thoroughly with methanol to provide 394 mg of the title product as a yellow solid. M.P.: 198-200° C. (Yield: 95%). ¹H NMR (400 MHz, CDCl₃, ppm): 8.02 (d, 2H, J=8.0 Hz); 7.61 (d, 4H, J=7.3 Hz); 7.42 (m, 8H); 7.28 (m, 6H); 4.32 (t, 2H, J=7.2 Hz); 1.94 (m, 2H); 1.41 (m, 6H); 0.94 (t, 3H, J=7.0 Hz). ¹³C NMR (100 MHz, CDCl₃, ppm): 141.72; 137.84; 135.36; 130.07; 128.96; 128.29; 127.70; 126.73; 122.77; 120.69; 118.05; 107.12; 43.22; 31.87; 29.23; 27.25; 22.85; 14.35.

### EXAMPLE 2

N-hexyl-2,7-bis(vinylene-(N-hexyl-2-carbazole))carbazole (37) (CCC): To a solution of compound 28 (500 mg, 1.25 mmol) and compound 13 (179 mg, 0.59 mmol) in anhydrous THF (25 mL) was slowly added potassium tert-butoxide (560 mg, 5.00 mmol, Aldrich Co.). The mixture was stirred at room temperature for 16 h and then poured into 100 mL of water. The aqueous layer was washed three times with chloroform and the combined organic layer was washed three times with water. The organic layer was dried over magnesium sulfate and concentrated under reduced pressure. The residue was purified by column chromatography (chloroform as eluent) followed by precipitation in cold methanol to provide 400 mg of the title product as a green solid. M.P.: 228-230° C. (Yield: 82%). ¹H NMR (400 MHz, CDCl₃, ppm): 8.08 (m, 6H); 7.54 (m, 8H); 7.46 (m, 8H); 7.23 (m, 2H); 4.35 (m, 6H); 1.93 (m, 6H); 1.40 (m, 18H); 0.90 (m, 9H). ¹³C NMR (100 MHz, CDCl₃, ppm): 141.78; 141.28; 141.14; 135.62; 135.60; 129.47; 129.41; 125.80; 123.02; 122.70; 122.68; 120.74; 120.64; 120.49; 119.13; 118.06; 117.81; 108.90; 106.97; 106.96; 43.28 (2C); 31.90; 31.86; 29.26; 29.22; 27.27; 27.24; 22.86; 22.82; 14.35; 14.30.

### EXAMPLE 3

5,5′-bis(vinylene-(N-hexyl-2-carbazole))-2,2′-bithiophene (38) (CTTC): To a solution of compound 28 (500 mg, 1.25 mmol) and 35 (133 mg, 0.58 mmol) in anhydrous THF (25 mL) was slowly added potassium tert-butoxide (560 mg, 5.00 mmol, Aldrich Co.). The mixture was stirred at room temperature for 16 h and then poured into 100 mL of water. The aqueous layer was washed three times with chloroform and the combined organic layer was washed three times with water. The organic layer was dried over magnesium sulfate and concentrated under reduced pressure. The residue was purified by column chromatography (chloroform as eluent) followed by precipitation in cold methanol to provide 217 mg of the title product as an orange solid. M.P.: 207-209° C. (Yield: 49%). ¹H NMR (400 MHz, CDCl₃, ppm): 8.07 (t, 4H, J=6.5 Hz); 7.43 (m, 8H); 7.27 (m, 4H); 7.13 (m, 4H); 7.03 (d, 2H, J=3.8 Hz); 4.32 (t, 4H, J=7.4 Hz); 1.90 (m, 4H); 1.38 (m, 12H); 0.89 (t, 6H, J=7.0 Hz). ¹³C NMR (100 MHz, CDCl₃, ppm): 142.55; 141.31; 141.05; 136.21; 134.78; 129.92; 127.26; 125.91; 124.33; 122.94; 122.88; 121.18; 120.78; 120.51; 119.17; 117.54; 108.91; 106.96; 43.29; 31.83; 29.19; 27.22; 22.80; 14.27.

### EXAMPLE 4

N-(2-ethylhexyl)-2,7-bis(vinylene-4-(1,1′-biphenylene))carbazole (BPCBP) (39): In a 25 mL flask, compound 20 (200 mg, 0.61 mmol), 4-bromo-1,1′-biphenyl (354 mg, 1.52 mmol, Aldrich Co.), palladium (II) acetate (5.50 mg, 0.02 mmol, Aldrich Co.), tri-o-tolylphosphine (37.0 mg, 0.12 mmol, Aldrich Co.) and degassed anhydrous DMF. (4 mL) were mixed under argon. The solution was heated at 80° C. followed by the addition of triethylamine (0.21 mL, 1.53 mmol, Aldrich Co.). The resulting solution was stirred at 110° C. under argon for 24 h. The mixture was cooled at room temperature and poured into water (150 mL). The aqueous layer was washed three times with chloroform (100 mL) and the combined organic fractions were dried with magnesium sulfate. The solvent was removed under reduced pressure and the crude green solid was completely dissolved in 150 mL of hot benzene. This solution was poured into methanol (300 mL) under vigorous stirring and the green precipitate was collected by filtration. The latter step was repeated twice to provide 263 mg of the title product as a green solid. M.P. >260° C. (Yield: 68%). ¹H NMR-(400 MHz, CDCl₃, ppm): 8.04 (d, 2H, J=8.6 Hz); 7.65 (m, 12H); 7.47 (m, 8H); 7.36 (m, 4H); 7.26 (m, 2H); 4.24 (m, 2H); 2.16 (m, 1H); 1.40 (m, 8H); 0.98 (t, 3H, J=7.3 Hz); 0.92 (t, 3H, J=7.2 Hz). ¹³C NMR (100 MHz, CDCl₃, ppm): 142.25; 140.91; 140.36; 136.85; 135.32; 130.11; 129.02; 127.77; 127.59; 127.52; 127.12; 127.09; 122.74; 120.62; 118.03; 107.41; 47.59; 39.61; 31.17; 29.01; 24.73; 23.31; 14.36; 11.24.

### EXAMPLE 5

N-hexyl-2,7-bis(cyanovinylenephenylene)carbazole (PCP-CN) (40): In a 25 mL flask, compound 13 (500 mg, 1.63 mmol), benzyl cyanide (457 mg, 3.90 mmol) and methanol (16 mL) were mixed under argon. A catalytic amount of potassium tert-butoxide was added and the solution was stirred at room temperature under argon for 24 h. The green-yellow precipitate formed during the reaction was filtered, rinsed with methanol and dried under reduced pressure to provide 722 mg of the title product as a bright green-yellow powder. M.P.: 126-128° C. (Yield: 88%). ¹H NMR (400 MHz, CDCl₃, ppm): 8.09 (m, 3H); 8.07 (s, 1H); 7.72 (m, 2H); 7.70 (m, 2H); 7.66 (s, 2H); 7.62 (m, 2H); 7.45 (m, 4H); 7.39 (m, 2H); 4.33 (t, 2H, J=7.3 Hz); 1.94 (m, 2H); 1.44 (m, 2H); 1.33 (m, 4H); 0.87 (t, 3H, J=7.1 Hz). ¹³C NMR (100 MHz, CDCl₃, ppm): 143.07; 141.72; 134.89; 132.05; 129.28; 129.25; 126.14; 124.30; 121.76; 121.27; 118.83; 110.70; 109.41; 43.65; 31.77; 29.22; 27.22; 22.78; 14.26.

### EXAMPLE 6

1,4-bis(vinylene-(N-hexyl-7-hexyl-2-carbazole))phenylene (H—CPC—H) (41): To a solution of compound 34 (1.03 g, 3.51 mmol) and 1,4-bis(methylphosphonate)benzene (0.53 g, 1.41 mmol) in anhydrous THF (15 mL) was added sodium tert-butoxide (0.54 g, 5.63 mmol). The resulting mixture was stirred under an argon atmosphere for 24 h at room temperature, which was followed by the addition of methanol (10 mL). The green-yellow precipitate so-obtained was collected by filtration, rinsed thoroughly with acetone and dried under reduced pressure for 24 h to provide 855 mg of the title product as a green-yellow solid (Yield=79%). M.P.: 280° C. (determined by DSC analysis at a scan rate of 10° C./minute). H—CPC—H was not soluble enough for NMR analysis.

### EXAMPLE 7

Poly(N-(2-ethylhexyl)-2,7-carbazolenevinylene) (PCV) by McMurry reaction²³: In a 100 mL flask, zinc powder (1.17 g, 17.9 mmol, Aldrich Co.) and anhydrous THF (15 mL) were mixed under argon. The resulting suspension was cooled to 0° C. in a ice/water bath and titanium (IV) chloride (1.70 g, 8.94 mmol, Aldrich Co.) was slowly added. The mixture was stirred at reflux for 1 h and then a solution of compound 12 (0.50 g, 1.49 mmol) in anhydrous THF (5 mL) was slowly added. The resulting solution was stirred for 24 h at reflux and then cooled to room temperature. An aqueous Na₂CO₃ solution (10%) was added and the resulting solution was stirred for 10 min. The precipitate was filtered, rinsed thoroughly with water, and then with methanol and washed in a soxhlet apparatus using acetone for 48 h to provide the title product as a yellow powder.

### EXAMPLE 8

Poly(N-(2-ethylhexyl)-2,7-carbazole-alt-2,5-dioctyloxy-1,4-phenylenevinylene) (PCVP) by Wittig reaction: In a 25 mL flask, compound 19 (1.00 g, 1.11 mmol), 2,5-dioctyloxy-1,4-diformylbenzene (434 mg, 1.11 mmol), anhydrous ethanol (4 mL) and anhydrous chloroform (6 mL) were mixed under argon and the resulting solution was cooled to 0° C. Sodium ethoxide (378 mg, 5.55 mmol) was slowly added and the solution was warmed to room temperature and stirred under argon for 24 h. The solution was poured into 200 mL of methanol and the precipitate was filtered, rinsed thoroughly with methanol and washed in a soxhlet apparatus using acetone for 48 h to provide the title product as an orange powder.

### EXAMPLE 9

Poly(N-(2-ethylhexyl)-2,7-carbazole-alt-2,5-dioctyloxy-1,4-phenylenevinylene) (PCVP) by Wittig-Horner reaction: In a 25 mL flask, compound 17 (571 mg, 0.99 mmol), 2,5-dioctyloxy-1,4-diformylbenzene (385 mg, 0.99 mmol) and anhydrous THF (10 mL) were mixed under argon. Potassium tert-butoxide (443 mg, 3.96 mmol) was slowly added and the solution was stirred at room temperature under argon for 24 h. The resulting solution was poured into 200 mL of methanol and the precipitate was filtered, rinsed thoroughly with methanol and washed in a soxhlet apparatus using acetone for 48 h to provide the title product as an orange solid having good film forming properties.

### EXAMPLE 10

Poly(N-(2-ethylhexyl)-2,7-carbazolenecyanovinylene-alt-2,5-dioctyloxy-1,4-phenylenevinylene) (PCCVP) by Knoevenagel reaction: In a 25 mL flask, compound 14 (250 mg, 0.70 mmol), 2,5-dioctyloxy-1,4-diformylbenzene (273 mg g, 0.70 mmol), anhydrous THF (4 mL) and anhydrous tert-butyl alcohol were mixed under argon. A catalytic amount of potassium tert-butoxide was added and the solution was stirred at room temperature under argon for 24 h. The resulting solution was poured into 200 mL of methanol and the precipitate was filtered, rinsed thoroughly with water, followed by rinsing with methanol and washing in a soxhlet apparatus using acetone for 48 h to provide the title product as a red solid having good film forming properties.

### EXAMPLE 11

Poly(N-(2-ethylhexyl-2,7-carbazolenevinylene-co-2,5-bis(diphenylamine)-1,4-phenylenevinylene-co-((4-(2-ethylhexyloxy)-phenyl)-bis-(4′-phenylene)amine) (PCVDPATA) by Wittig-Horner reaction: In a 25 mL flask, compound 17 (343 mg, 0.60 mmol), 2,5-bis(diphenylamino)terephthaldicarboxaldehyde (139 mg, 0.30 mmol), [4-(2-ethylhexyloxy)-phenyl-bis-(4′-formylphenyl) (127 mg, 0.30 mmol) and anhydrous THF (12 mL) were mixed under argon. Potassium tert-butoxide (265 mg, 2.37 mmol) was slowly added and the solution was stirred at room temperature under argon for 24 h. The resulting solution was poured into 200 mL of methanol and the orange precipitate was filtered, rinsed thoroughly with methanol and washed in a soxhlet apparatus using acetone for 48 h to provide the title product as an orange solid having good film forming properties.

### EXAMPLE 12

Poly(N-(2-ethylhexyl-2,7-carbazolenecyanovinylene-co-2,5-bis(diphenylamine)-1,4-phenylenecyanovinylene-co-2,5-dioctyloxy-1,4-phenylenecyanovinylene) (PCVDPAP) by Knoevenagel reaction: In a 25 mL flask, compound 14 (250 mg, 0.70 mmol), 2,5-bis(diphenylamino)terephthaldicarboxaldehyde (164 mg, 0.35 mmol), 2,5-dioctyloxy-1,4-diformylbenzene (137 mg, 0.35 mmol), anhydrous THF (5 mL) and anhydrous tert-butyl alcohol (5 mL) were mixed under argon. A catalytic amount of potassium tert-butoxide was added and the solution was stirred at room temperature under argon for 24 h. The resulting solution was poured into 200 mL of methanol and the precipitate was filtered, rinsed thoroughly with water followed by methanol and washed in a soxhlet apparatus using acetone for 48 h to provide the title product as a red solid having good film forming properties.

### EXAMPLE 13

Poly(N-(2-ethylhexyl-2,7-carbazolenevinylene-alt-6,6′-(2,2′-bis(2″-ethylhexyloxy)-1,1′-binaphatylene) (PCVBN) by Heck reaction: In a 25 mL flask, compound 20 (200 mg, 0.61 mmol), 6,6′-dibromo-2,2′-bis(2″-ethylhexyloxy)-1,1′-binaphthyl (406 mg, 0.61 mmol, Aldrich Co.), palladium (II) acetate (14.0 mg, 0.06 mmol, Aldrich Co.), tetrabutylammonium chloride (202 mg, 0.61 mmol, Aldrich Co.), freshly dried lithium chloride (26.0 mg, 0.61 mmol), anhydrous potassium carbonate (168 mg, 1.22 mmol) and degassed anhydrous DMF (18 mL) were mixed under argon. The solution was heated at 120° C. and stirred under argon for 72 h. The resulting solution was poured into 200 mL of cold methanol and the precipitate was filtered, rinsed thoroughly with water followed by methanol and washed in a soxhlet apparatus using acetone for 48 h to provide the title product as a yellow solid.

### EXAMPLE 14

Poly[(N-(4-octyloxyphenyl))-2,7-carbazolenevinylene-alt-(3-hexyl-2,5-thiophenevinylene)] (PPCVT) by Horner-Emmons reaction: In a 25 mL flask, compound 22 (412 mg, 0.96 mmol), 3-hexyl-2,5-bis(methylphosphonate)thiophene (452 mg, 0.96 mmol) and anhydrous THF (11 mL) were mixed under argon. Potassium tert-butoxide (471 mg, 3.85 mmol) was slowly added and the solution was stirred at room temperature under argon for 24 h. The resulting solution was poured into 200 mL of methanol and the orange precipitate was filtered, rinsed thoroughly with methanol and washed in a soxhlet apparatus using acetone for 48 h to provide the title product as an red solid having good film forming properties.

Although the present invention has been described hereinabove by way of preferred embodiments thereof, it can be modified, without departing from the spirit and nature of the subject invention as defined in the appended claims.

### REFERENCES

1. Handbook of Conducting Polymers, 2^{nd} ed.; Skotheim, T. A.; Elsenbaumer, R. L.; Reynolds, J. R.; Eds. Marcel Dekker, New York, 1998.
2. Leclerc, M.; Faïd, K. Adv. Mater. 1997, 9, 1087; Leclerc, M. Adv. Mater. 1999, 11, 1491; McQuade, D. T.; Pullen, A. E.; Swager, T. M. Chem. Rev. 2000, 100, 2537; Ho, H. A.; Boissinot, M.; Bergeron, M. G.; Corbeil, G.; Doré, K.; Boudreau, D.; Leclerc, M. Angew. Chem. Int. Ed. 2002, 41, 1548.
3. Burroughes, J. H.; Bradley, D. D. C.; Brown, A. R.; Marks, R. N.; MacKay, K.; Friend, R. H.; Burns, P. L.; Holmes, A. B. Nature 1990, 347, 539.
4. Mitschke, U.; Bauerle, P. J. Mater. Chem. 2000, 10, 1471.
5. Kraft, A.; Grimsdale, A. C.; Holmes, A. B. Angew. Chem. Int. Ed. 1998, 37, 402.
6. Bernius, M. T.; Inbasekaran, M.; O'Brien, J.; Wu, W. Adv. Mater. 2000, 12, 1737; Leclerc, M. J. Polym. Sci., Polym. Chem. 2001, 39, 2867; Neher, D. Macromol. Rapid Commun. 2001, 22, 1365.
7. Brabec, C. J.; Sariciftci, N. S.; Hummelen, J. C. Adv. Funct. Mater. 2002, 12, 192; Sariciftci, N. S.; Braun, D.; Zhang, C.; Srdanov, V. I.; Heeger, A. J.; Stucky, G.; Wudl, F. Appl. Phys. Lett. 1993, 62, 585.
8. Morin, J.-F.; Leclerc, M. Macromolecules 2001, 34, 4680.
9. Zotti, G.; Schiavon, G.; Zecchin, S.; Morin, J.-F.; Leclerc, M. Macromolecules 2002, 35, 2122.
10. Morin, J.-F.; Leclerc, M.; Macromolecules 2002, 35, 8413.
11. Morin, J.-F.; Beaupré, S.; Leclerc, M.; Lëvesque, I.; D'Iorio, M. Appl. Phys. Lett. 2002, 80, 341.
12. Drolet, N.; Beaupré, S.; Morin, J.-F.; Tao, Y.; Leclerc, M. J. Opt. A: Pure Appl. Opt, 2002, 4, S252.
13. DiCesare, N.; Belletête, M.; Leclerc, M.; Durocher, G. Chem. Phys. Lett. 1998, 291, 487; Donat-Bouillud, A. Ph. D. Thesis, 1998, Université de Montréal.
14. Pavlopoulos, T. G.; Boyer, J. H.; Shah, M.; Thangaraj, K.; Soong, M. L. Appl. Opt 1990, 29, 3885.
15. Shi, J.; Zheng, S. Macromolecules 2001, 34 (19), 6571.
16. Kim, S. W.; Shim, S. C.; Jung, B.-J.; Shim, H.-K. Polymer 2002, 43, 4297.
17. Sarker, A. M.; Ding, L.; Lahti, P. M.; Karasz, F. E. Macromolecules 2002, 35, 223.
18. Zheng, L.; Urian, R. C.; Liu, Y.; Jen, A. K.-Y.; Pu, L. Chem. Mater. 2000, 12, 13.
19. Chen, Y.; Huang, Y. Y.; Wu, T. Y. J. Polym. Sci. Part A 2002, 40, 2927.
20. Chaudhary, S. K.; Hernandez, O. Tet. Lett. 1979, 2, 95.
21. Liu, B.; Yu, W.-L.; Pei, J.; Liu, S.-Y.; Lai, Y.-H.; Huang, W. Macromolecules 2001, 34 (23), 7932.
22. Gòmez, R.; Segura, J. L.; Martin, N. J. Org. Chem. 2000, 65, 7501.
23. Goldoni, F.; Janssen, A. J.; Meijer, E. W. J. Polym. Sci. Part A 1999, 37, 4629.

What is claimed is:

1. A compound of Formula I: 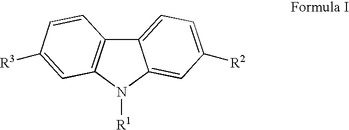
wherein:
R¹ is selected from the group consisting of H, alkyl, and aryl;
R² and R³ are independently selected from the group consisting of H, alkyl, formyl, hydroxymethyl, trityloxymethyl, acetonitrile, chloromethyl, methylphosphonate, methyltriphenylphosphonium and vinyl.

2. A compound as defined in claim 1, selected from the group consisting of: 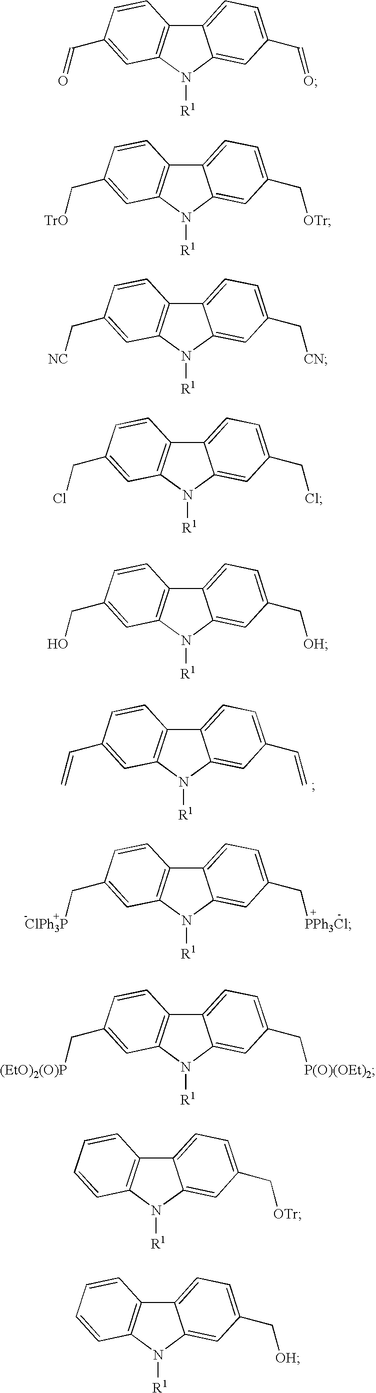 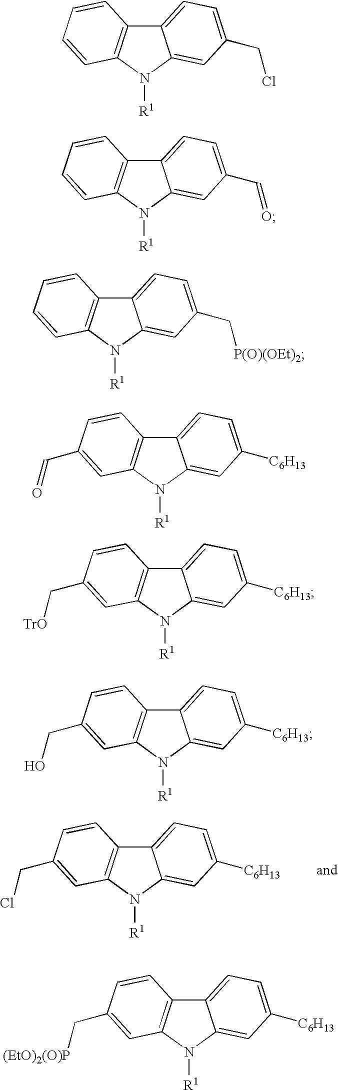
wherein R¹ is as previously defined.

3. A compound as defined in claim 1, selected from the group consisting of: 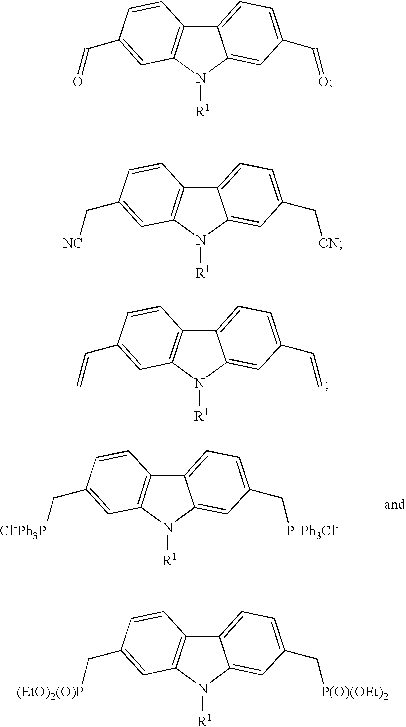
wherein R¹ is as previously defined.

4. A compound as defined in claims 2 and 3 having the formula: 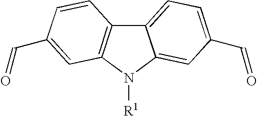
wherein R¹ is alkyl.

5. A compound as defined in claim 4, wherein R¹ is hexyl or 2-ethylhexyl.

6. A compound as defined in claim 4, wherein R¹ is aryl.

7. A compound as defined in claim 4, wherein R¹ is 4-octyloxyphenyl.

8. A compound as defined in claims 2 and 3 having the formula: 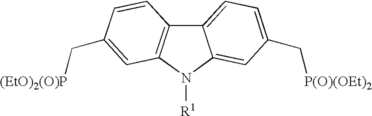
wherein R¹ is alkyl.

9. A compound as defined in claim 8, wherein R¹ is hexyl or 2-ethylhexyl.

10. A compound as defined in claims 2 and 3 having the formula: 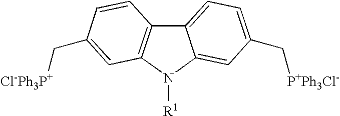
wherein R¹ is alkyl.

11. A compound as defined in claim 10, wherein R¹ is 2-ethylhexyl.

12. A compound as defined in claims 2 and 3 having the formula: 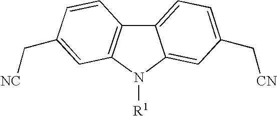
wherein R¹ is alkyl.

13. A compound as defined in claim 12, wherein R¹ is 2-ethylhexyl.

14. A compound as defined in claims 2 and 3 having the formula: 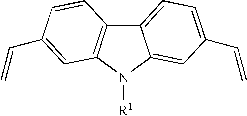
wherein R¹ is alkyl.

15. A compound as defined in claim 14, wherein. R¹ is 2-ethylhexyl.

16. A compound as defined in claim 2 having the formula: 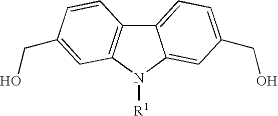
wherein R¹ is alkyl.

17. A compound as defined in claim 16, wherein R¹ is hexyl or 2-ethylhexyl.

18. A compound as defined in claim 2 having the formula: 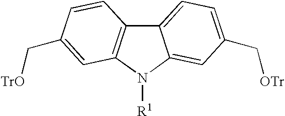
wherein R¹ is H or alkyl.

19. A compound as defined in claim 18, wherein R¹ is hexyl or 2-ethylhexyl.

20. A compound as defined in claim 18, wherein R¹ is aryl.

21. A compound as defined in claim 20, wherein R¹ is 4-octyloxyphenyl.

22. A compound as defined in claim 2 having the formula: 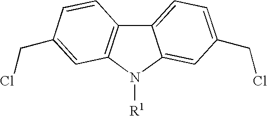
wherein R¹ is alkyl.

23. A compound as defined in claim 22, wherein R¹ is hexyl.

24. A compound as defined in claim 2 having the formula: 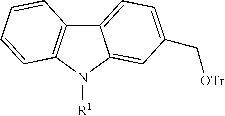
wherein R¹ is H or alkyl.

25. A compound as defined in claim 24, wherein R¹ is hexyl.

26. A compound as defined in claim 2 having the formula: 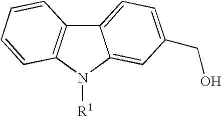
wherein R¹ is alkyl.

27. A compound as defined in claim 26, wherein R¹ is hexyl.

28. A compound as defined in claim 2 having the formula: 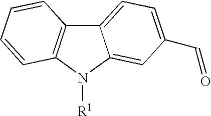
wherein R¹ is alkyl.

29. A compound as defined in claim 28, wherein R¹ is hexyl.

30. A compound as defined in claim 2 having the formula: 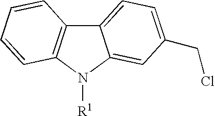
wherein R¹ is alkyl.

31. A compound as defined in claim 30, wherein R¹ is hexyl.

32. A compound as defined in claim 2 having the formula: 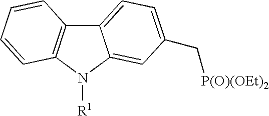
wherein R¹ is alkyl.

33. A compound as defined in claim 32, wherein R¹ is hexyl.

34. A compound as defined in claim 2 having the formula: 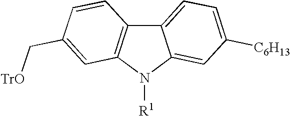
wherein R¹ is H or alkyl.

35. A compound as defined in claim 34, wherein R¹ is methyl.

36. A compound as defined in claim 2 having the formula: 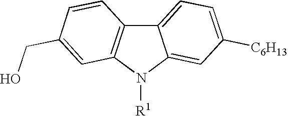
wherein R¹ is alkyl.

37. A compound as defined in claim 36, wherein R¹ is methyl.

38. A compound as defined in claim 2 having the formula: 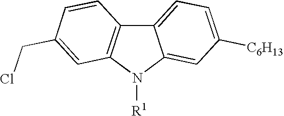
wherein R¹ is alkyl.

39. A compound as defined in claim 38, wherein R¹ is methyl.

40. A compound as defined in claim 2 having the formula: 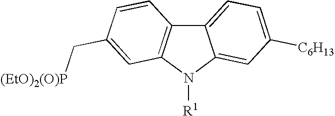
wherein R¹ is alkyl.

41. A compound as defined in claim 40, wherein R¹ is methyl.

42. A compound as defined in claim 2 having the formula: 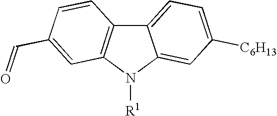
wherein R¹ is alkyl.

43. A compound as defined in claim 42, wherein R¹ is methyl.

44. An oligomer comprising the reaction product of a first compound of Formula I and at least a second compound, said second compound being either a compound of Formula I; benzaldehyde; 5,5′-diformyl-2-2′bithiophene, 4-bromo-1,1′biphenyl; benzyl cyanide; or 1,4-bis(methylphosphonate)benzene.

45. An oligomer as defined in claim 44 having the formula: 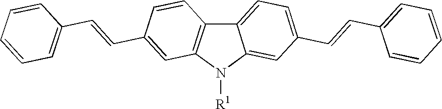
wherein R¹ is selected from the group consisting of H, alkyl, and aryl.

46. An oligomer as defined in claim 45, wherein R¹ is alkyl.

47. An oligomer as defined in claim 46, wherein R¹ is hexyl or 2-ethyl hexyl.

48. An oligomer as defined in claim 47, wherein R¹ is hexyl.

49. An oligomer as defined in claim 45 wherein the first compound of Formula I is of the formula: 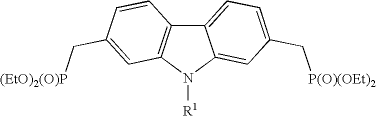
wherein R¹ is selected from the group consisting of H, alkyl, and aryl.

50. An oligomer as defined in claim 49, wherein R¹ is alkyl.

51. An oligomer as defined in claim 50, wherein R¹ is hexyl or 2-ethylhexyl.

52. An oligomer as defined in claim 51, wherein R¹ is hexyl.

53. An oligomer as defined in any one of claims 45 to 52, wherein the second compound is benzaldehyde.

54. An oligomer as defined in claim 44 having the formula: 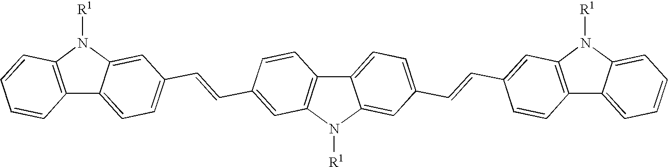
wherein R¹ is selected from the group consisting of H, alkyl, and aryl.

55. An oligomer as defined in claim 54, wherein R¹ is alkyl.

56. An oligomer as defined in claim 55, wherein R¹ is hexyl or 2-ethylhexyl.

57. An oligomer as defined in claim 56, wherein R¹ is hexyl.

58. An oligomer as defined in claim 54 wherein the first compound of Formula I is of the formula: 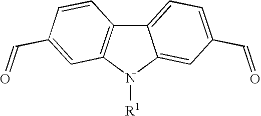
wherein R¹ is selected from the group consisting of H, alkyl, and aryl.

59. An oligomer as defined in claim 58, wherein R¹ is alkyl.

60. An oligomer as defined in claim 59, wherein R¹ is hexyl or 2-ethylhexyl.

61. An oligomer as defined in claim 59, wherein R¹ is hexyl.

62. An oligomer as defined in claim 58, wherein R¹ is aryl.

63. An oligomer as defined in claim 62, wherein R¹ is 4-octyloxyphenyl.

64. An oligomer as defined in claim 54 wherein the second compound of Formula I is of the formula: 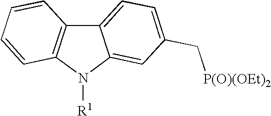
wherein R¹ is selected from the group consisting of H, alkyl, and aryl.

65. An oligomer as defined in claim 64, wherein R¹ is alkyl.

66. An oligomer as defined in claim 65, wherein R¹ is hexyl.

67. An oligomer as defined in claim 44 having the formula: 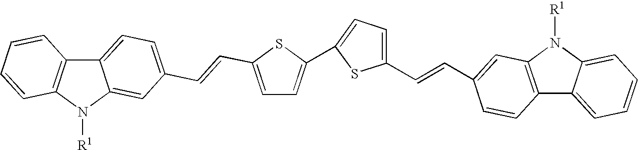
wherein R¹ is selected from the group consisting of H, alkyl, and aryl.

68. An oligomer as defined in claim 67, wherein R¹ is alkyl.

69. An oligomer as defined in claim 68, wherein R¹ is hexyl.

70. An oligomer as defined in claim 67 wherein the first compound of Formula I is of the formula: 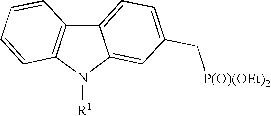
wherein R¹ is selected from the group consisting of H, alkyl, and aryl.

71. An oligomer as defined in claim 70, wherein R¹ is alkyl.

72. An oligomer as defined in claim 71, wherein R¹ is hexyl.

73. An oligomer as defined in any one of claims 67 to 72, wherein the second compound is 5,5′-diformyl-2-2′bithiophene.

74. An oligomer as defined in claim 44 having the formula: 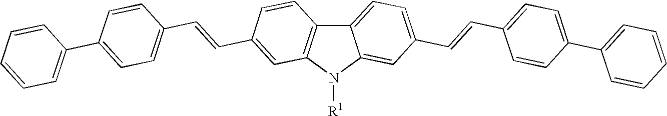
wherein R¹ is selected from the group consisting of H, alkyl, and aryl.

75. An oligomer as defined in claim 74, wherein R¹ is alkyl.

76. An oligomer as defined in claim 75, wherein R¹ is 2-ethylhexyl.

77. An oligomer as defined in claim 74 wherein the first compound of Formula I is of the formula: 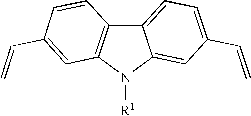
wherein R¹ is selected from the group consisting of H, alkyl, and aryl.

78. An oligomer as defined in claim 77, wherein R¹ is alkyl.

79. An oligomer as defined in claim 78, wherein R¹ is 2-ethylhexyl.

80. An oligomer as defined in any one of claims 74 to 79, wherein the second compound is 4-bromo-1,1′biphenyl.

81. An oligomer as defined in claim 44 having the formula: 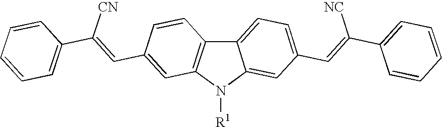
wherein R¹ is selected from the group consisting of H, alkyl, and aryl.

82. An oligomer as defined in claim 81, wherein R¹ is alkyl.

83. An oligomer as defined in claim 82, wherein R¹ is hexyl or 2-ethylhexyl.

84. An oligomer as defined in claim 81, wherein R¹ is aryl.

85. An oligomer as defined in claim 84, wherein R¹ is 4-octyloxyphenyl.

86. An oligomer as defined in claim 81 wherein the first compound of Formula I is of the formula: 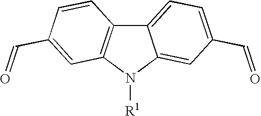
wherein R¹ is selected from the group consisting of H, alkyl, and aryl.

87. An oligomer as defined in claim 86, wherein R¹ is alkyl.

88. An oligomer as defined in claim 87, wherein R¹ is hexyl or 2-ethylhexyl.

89. An oligomer as defined in claim 88, wherein R¹ is hexyl.

90. An oligomer as defined in claim 86, wherein R¹ is aryl.

91. An oligomer as defined in claim 90, wherein R¹ is 4-octyloxyphenyl.

92. An oligomer as defined in any one of claims 81 to 91, wherein the second compound is benzyl cyanide.

93. An oligomer as defined in claim 44 having the formula: 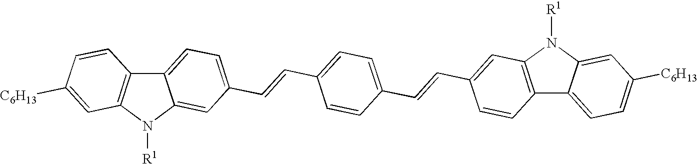
wherein R¹ is selected from the group consisting of H, alkyl, and aryl.

94. An oligomer as defined in claim 93, wherein R¹ is alkyl.

95. An oligomer as defined in claim 94, wherein R¹ is methyl.

96. An oligomer as defined in claim 93, wherein the first compound of Formula I is of the formula: 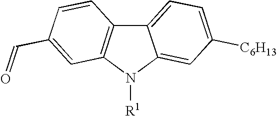
wherein R¹ is selected from the group consisting of H, alkyl, and aryl.

97. An oligomer as defined in claim 96, wherein R¹ is alkyl.

98. An oligomer as defined in claim 97, wherein R¹ is methyl.

99. An oligomer as defined in any one of claims 93 to 98, wherein the second compound is 1,4-(bis)methylphosphonate)benzene.

100. A polymer comprising the reaction product of a compound of Formula I and optionally at least one compound selected from the group consisting of 2,5-dioctyloxy-1,4-diformylbenzene; 2,5-bis(diphenylamino)terephthaldicarboxaldehyde; {4-(2-ethylhexyloxy)-phenyl]-bis-(4′formylphenyl); 6,6′-dibromo-2,2′-bis(2″-ethylhexyloxy)-1,1′-binaphthyl; and 3-hexyl-2,5-bis(methylphosphonate)thiophene.

101. A polymer as defined in claim 100, comprising monomeric groups of the formula: 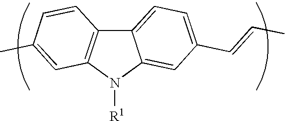
wherein R¹ is selected from the group consisting of H, alkyl, and aryl.

102. A polymer as defined in claim 101, wherein R¹ is alkyl.

103. A polymer as defined in claim 102, wherein R¹ is hexyl or 2-ethylhexyl.

104. A polymer as defined in claim 103, wherein R¹ is 2-ethylhexyl.

105. A polymer as defined in claim 104 having the formula: 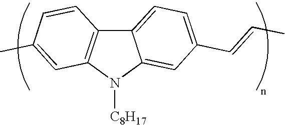
wherein “n” is an integer ranging from 5 to 100.

106. A polymer as defined in claim 100, comprising monomeric groups of the formula: 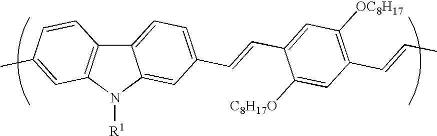
wherein R¹ is selected from the group consisting of H, alkyl, and aryl.

107. A polymer as defined in claim 106, wherein R¹ is alkyl.

108. A polymer as defined in claim 107, wherein R¹ is hexyl or 2-ethylhexyl.

109. A polymer as defined in claim 108, wherein R¹ is 2-ethylhexyl.

110. A polymer as defined in claim 109 having the formula: 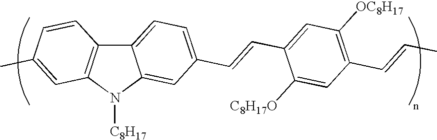
wherein “n” is an integer ranging from 5 to 100.

111. A polymer as defined in claim 100, comprising monomeric groups of the formula: 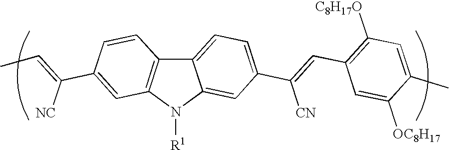
wherein R¹ is selected from the group consisting of H, alkyl, and aryl.

112. A polymer as defined in claim 111, wherein R¹ is alkyl.

113. A polymer as defined in claim 112, wherein R¹ is hexyl or 2-ethylhexyl.

114. A polymer as defined in claim 113, wherein R¹ is 2-ethylhexyl.

115. A polymer as defined in claim 114 having the formula: 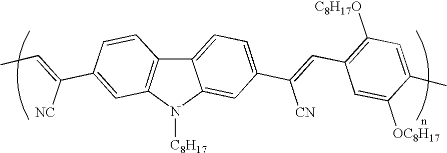
wherein “n” is an integer ranging from 5 to 100.

116. A polymer as defined in claim 100, comprising monomeric groups of the formula: 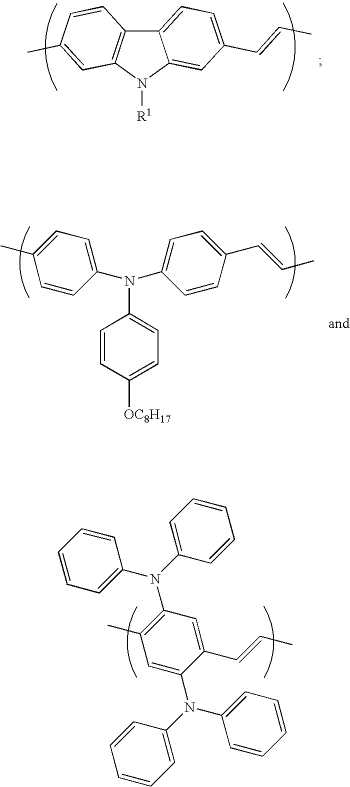
wherein R¹ is selected from the group consisting of H, alkyl, and aryl.

117. A polymer as defined in claim 116, wherein R¹ is alkyl.

118. A polymer as defined in claim 117, wherein R¹ is hexyl or 2-ethylhexyl.

119. A polymer as defined in claim 118, wherein R¹ is 2-ethylhexyl.

120. A polymer as defined in claim 119 having the formula: 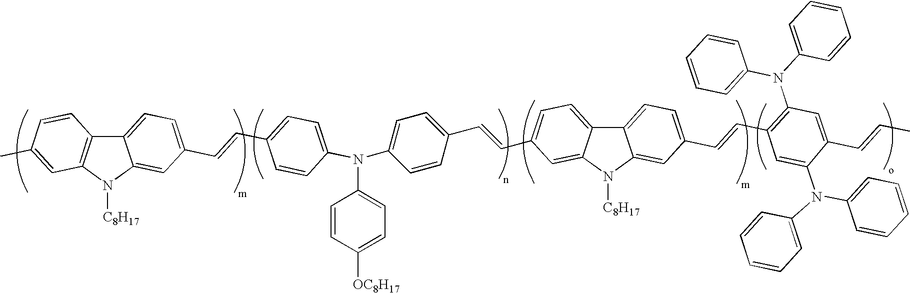
wherein “n”, “m”, and “o” are integers ranging from 5 to 100.

121. A polymer as defined in claim 100, comprising monomeric groups of the formula: 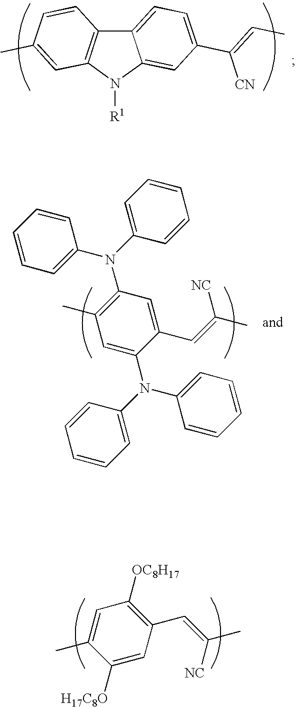
wherein R¹ is selected from the group consisting of H, alkyl, and aryl.

122. A polymer as defined in claim 121, wherein R¹ is alkyl.

123. A polymer as defined in claim 122, wherein R¹ is hexyl or 2-ethylhexyl.

124. A polymer as defined in claim 123, wherein R¹ is 2-ethylhexyl.

125. A polymer as defined in claim 124 having the formula: 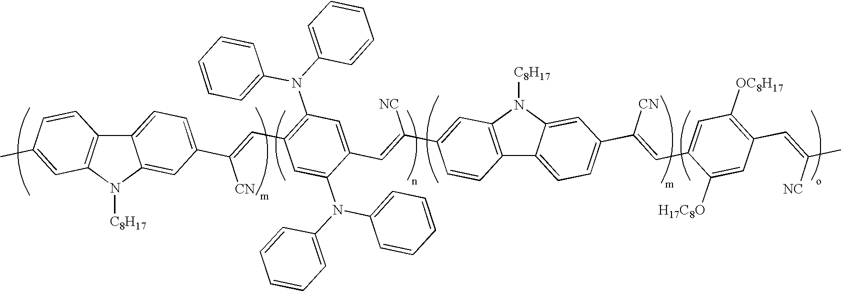
wherein “n”, “m”, and “o” are integers ranging from 5 to 100.

126. A polymer as defined in claim 100, comprising monomeric groups of the formula: 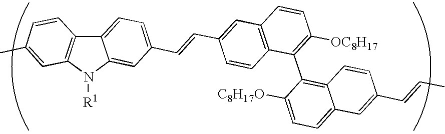
wherein R¹ is selected from the group consisting of H, alkyl, and aryl.

127. A polymer as defined in claim 126, wherein R¹ is alkyl.

128. A polymer as defined in claim 127, wherein R¹ is hexyl or 2-ethylhexyl.

129. A polymer as defined in claim 128, wherein R¹ is 2-ethylhexyl.

130. A polymer as defined in claim 129 having the formula: 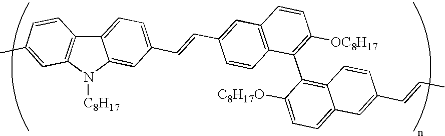
wherein “n” is an integer ranging from 5 to 100.

131. A polymer as defined in claim 100, comprising monomeric groups of the formula: 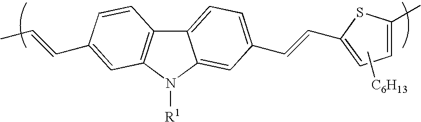
wherein R¹ is selected from the group consisting of H, alkyl, and aryl.

132. A polymer as defined in claim 131, wherein R¹ is aryl.

133. A polymer as defined in claim 132, wherein R¹ is 4-octyloxyphenyl.

134. A polymer as defined in claim 133 having the formula: 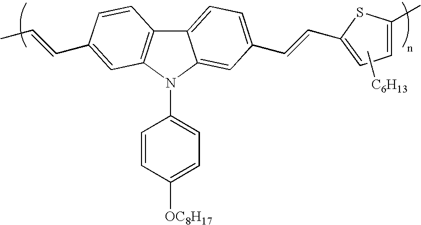
wherein “n” is an integer ranging from 5 to 100.

135. A composition comprising the oligomer and/or polymer of claims 44-134.

136. An electronic device comprising a film or coating comprising the oligomer and/or polymer of claims 44-134.

137. The electronic device of claim 136, configured as a light-emitting diode.

138. The electronic device of claim 136, configured as a field-effect transistor.

139. The electronic device of claim 136, configured as a solar cell.
